(19) United States
(12) Patent Application Publication
(10) Pub. No.: US 2010/0173332 A1
(43) Pub. Date: Jul. 8, 2010

(54) Method for the Fluorescent Detection of Nitroreductase Activity Using Nitro-Substituted Aromatic Compounds

(75) Inventors: Jeffrey B. Smaill, Auckland (NZ); Adam V. Patterson, Waiheke Island (NZ); Dean C. Singleton, Albany (NZ)

Correspondence Address: OCCHIUTI ROHLICEK & TSAO, LLP, 10 FAWCETT STREET, CAMBRIDGE, MA, 02138, US

(73) Assignee: Auckland Uniservices Limited

(21) Appl. No.: 12/440,267

(22) PCT Filed: Sep. 7, 2007
(86) PCT No.: PCT/NZ2007/000262

§ 371 Date: Dec. 7, 2009

(30) Foreign Application Priority Data
Sep. 7, 2006 (NZ) 549711

(51) Int. Cl. G01N 33/567; C07C 69/76

(52) U.S. Cl. 435/7.21; 560/9

(57) ABSTRACT

A method utilising one or more fluorogenic probes, for the detection of nitroreductase activity. The non-fluorescent probes are reduced in the presence of nitroreductase to form fluorescent derivatives that may be detected using fluorescence spectroscopy. In particular, the method may be used to detect and/or identify a plurality of nitroreductase in a single test environment

This invention relates generally to a method for the fluorescent detection of nitroreductase activity using at least one fluorogenic probe. The method utilises one or more probes, which are non-fluorescent aromatic compounds containing at least one NO₂ group, that is reduced to NHOH or NH₂ by the action of a nitroreductase resulting in the production of a strongly fluorescent molecule. In particular, the invention relates to a method of detection based on the use of a plurality of such probes in a common environment. A novel class of nitro-substituted compounds is also provided.

### BACKGROUND

A small group of non-fluorescent 7-nitrocoumarins are described in US20020031795A1 that are reduced by nitroreductase into a fluorescent derivative for the fluorogenic detection of microbial infection. The present inventors have found that the 7-nitrocoumarins are activated in wild type neoplastic mammalian cells and tend to lack stability in solution over a period of time. The inventors have also established that the intensity of the fluorescent signal of the 7-nitrocoumarins can be variable making it very difficult to undertake quantitative diagnostic studies.

A 6-chloro-9-nitro-5H-benzo[a]phenoxazin-5-one (C-22220, CNOB) has been described as a fluorogenic probe for nitroreductase activity, but no specific information on particular reductases is given (Molecular Probes Handbook, 10^{th} Edition, page 535). The inventors have established that this compound lacks stability in culture medium under conditions of low oxygen making it unsuitable as a probe for mammalian single-electron reductases which require anaerobic conditions for activity.

Some nitroacridone derivatives are described in US20040191792A1 as dyes having characteristic fluorescence lifetimes. These acridone dyes do not require reduction by a nitroreductase to exhibit fluorescence. The acridone chromophore is inherently highly fluorescent.

Nitro quenched cyanine dyes are taught in US20030186348A1 as a way of enhancing the fluorescence output of the cyanine dyes for the detection of microbial nitroreductases with the emphasis on reporter gene applications. These compounds have considerable fluorescence in their quenched form in cell culture and upon the action of a nitroreductase increase in fluorescence by three to four-fold offering a limited dynamic range for reporter gene applications.

The ability to assay for more than one reporter enzyme is particularly inefficient and laborious due to the current inability to identify multiple reporter enzymes in a common test environment. For example firefly or renilla luciferases may only be quantified in cultured mammalian cells following cell lysis (destruction) a procedure that is incompatible with many other assays. Multiplexing unrelated reporter genes is usually problematic, or at best requires each to be assayed separately employing different chemistry and detection methods, with sequential measurements and iterative chemistry steps or sample replating. In contrast, nitroreductase detection is a non-destructive assay protocol that can be detected in a common environment, for example an individual well or a single cell, using the same detection method simultaneously (e.g. fluorescence emission), without the need to quench one before measuring another. Nitroreductases are thus ideal for multiplexing on sub-cellular imaging systems including microscopes, sub-cellular imagers and plate readers.

It is an object of the invention to provide a method for the fluorescent detection of nitroreductase activity using nitro-substituted aromatic compounds as one or more fluorogenic probes to assay, qualify and/or quantify for diagnostic purposes, including the detection of microbial infection, the use in imaging applications, quantification of microorganisms in test samples, diagnostic testing for human reductase activity and detection in reporter gene assays, or to at least provide the public with a useful choice. Another object of the invention is to provide a novel class of nitro-substituted aromatic compounds, or to at least provide a useful alternative.

### SUMMARY OF THE INVENTION

In a first aspect, the present invention provides a method for the fluorescent detection of nitroreductase activity using a fluorogenic probe suitable for use as a nitroreductase probe, the method including the step of applying a plurality of probes to a sample and monitoring for the presence of at least one nitroreductase enzyme in a common test environment.

Preferably, the step of monitoring for the presence of at least one nitroreductase includes the step of monitoring for the presence of a reduced fluorescent derivative of the fluorogenic probe.

Preferably, the reduced derivatives may be excited at predetermined wavelengths in the UV/visible range and the fluorescence emission determined.

Preferably, the excitation wavelength will be between 200-700 nm. More preferably, the excitation wavelength may be selected from 295, 340, 355, 405, 440 and 485 nm.

Preferably, the fluorescence emission will be in the UV/visible/IR range.

Preferably, the fluorescence emission wavelength will be between 300-800 nm. More preferably, the fluorescence emission wavelength may be selected from 370, 460, 510, 535, 540 and 585 nm.

Preferably, the method further includes the step of quantifying the activity of at least one nitroreductase. More preferably, the step of quantifying the nitroreductase activity includes the step of quantifying the formation of the fluorescent derivative from the fluorescence emission intensity.

Preferably, the monitoring of a plurality of fluorogenic probes can be performed simultaneously.

Preferably, the monitoring of a plurality of fluorogenic probes can be performed sequentially.

In a second aspect the present invention provides a method for the fluorescent detection of nitroreductase activity using as one or more fluorogenic probes a class of nitro-aromatic compounds of Formula I-V as defined herein, suitable for use as a nitroreductase probe, the method including the step of applying the one or more fluorogenic probes to a sample and monitoring for the presence of at least one nitroreductase.

Preferably, the step of monitoring for the presence of nitroreductase activity includes the step of monitoring for the presence of a reduced fluorescent derivative of a compound of Formula I-V.

Preferably, the method further includes the step of quantifying the nitroreductase activity. More preferably, the step of quantifying the nitroreductase activity includes the step of quantifying the formation of the fluorescent derivative from the fluorescence emission intensity.

Preferably, the method uses a plurality of fluorogenic probes.

Preferably, the monitoring can be performed in a common test environment.

In a third aspect the present invention provides a method for the fluorescent detection of nitroreductase activity using as one or more fluorogenic probes a class of nitro-aromatic compounds of Formula I suitable for use as a nitroreductase probe

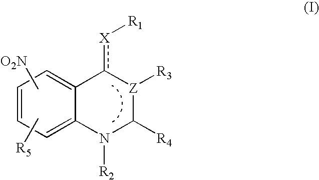

wherein X represents N, NH, NR⁶, O or S;
Z represents C, CH or N;
wherein R¹, if present, may be selected from H, R⁷, (CR⁷R⁸)ₙCOOH, (CR⁷R⁸)ₙCOOR⁹, (CR⁷R⁸)ₙCONH₂, (CR⁷R⁸)ₙCONHR⁹, (CR⁷R⁸)ₙCONR⁹R¹⁰, (CR⁷R⁸)ₙOH, (CR⁷R⁸)ₙOR⁹, (CR⁷R⁸)ₙOPO(OH)₂, COOH, COOR⁷, (CR⁷R⁸)ₙNR⁹R¹⁰, (CR⁷R⁸)ₙ-morpholinyl, (CR⁷R⁸)ₙ-piperazinyl; (CR⁷R⁸)ₙ-1-methylpiperazinyl; (CR⁷R⁸)ₙ-piperidinyl; (CR⁷R⁸)ₙ-pyrrolidinyl or (CR⁷R⁸)ₙ-imidazolyl;
wherein R² may represent H, R¹¹, (C′R¹¹R¹²)ₙCOO(C″R¹¹R¹²)ₙNR¹³R¹⁴, (C′R¹¹R¹²)ₙCONH(C″R¹¹R¹²)ₙNR¹³R¹⁴, wherein C′ and C″ may be optionally and independently substituted with C₁-C₆ alkyl and/or OH;
R³ and R⁴ may independently represent H, R¹⁵, Ar, —(CH═CH)ₙAr; SO₃H, CN
wherein Ar may represent a substituted or unsubstituted phenyl, pyridyl, pyrimidinyl, thiazolyl, oxazoylyl, imidazolyl, furanyl, pyrrolyl, benzoxazolyl, benzthiazolyl, benzofuranyl, indolyl, indazolyl, benzimdazolyl, wherein each Ar is optionally substituted with NO₂, R¹⁶, OH, OR¹⁶, SH, SR¹⁶, halogen, CF₃, NH₂, NHR¹⁶, NR¹⁶R¹⁷, NHCOR¹⁶, NR¹⁶COR¹⁷, NHCOOR¹⁶, NR¹⁶COOR¹⁷, (CR¹⁶R¹⁷)ₙCOOH, (CR¹⁶R¹⁷)ₙCOOR¹⁸, (CR¹⁶R¹⁷)ₙCONH₂, (CR¹⁶R¹⁷)ₙCONHR¹⁸, (CR¹⁶R¹⁷)ₙCONR¹⁸R¹⁹, (CR¹⁶R¹⁷)ₙOH, (CR¹⁶R¹⁷)ₙOR¹⁸, (CR¹⁶R¹⁷)ₙOPO(OH)₂, COOH, COOR¹⁶, CONH₂, CONHR¹⁶, CONR¹⁶R¹⁷, COR¹⁶, CN, SOR¹⁶, SO₂R¹⁶, SO₂NR¹⁶R¹⁷, SO₃H, or when Z represents C, R³ and R⁴ may together form a fused aromatic ring optionally substituted at one or more of the available carbons with a C₁-C₆ alkyl, halogen, SO₃H or CN;
wherein R⁵ may represent H, C₁-C₆ alkyl, halogen, CN, NO₂, Ar, —(CH═CH)ₙAr, COR²⁰, SOR²⁰, SO₂R²⁰, CO(CR²⁰R²¹)ₙOH, SO(CR²⁰R²¹)ₙOH, SO₂(CR²⁰R²¹)ₙOH, CO(CR²⁰R²¹)ₙCOOR²², SO(CR²⁰R²¹)ₙCOOR²², SO₂(CR²⁰R²¹)ₙCOOR²², CO(CR²⁰R²¹)ₙNR²²R²³, SO(CR²⁰R²¹)ₙNR²²R²³, SO₂(CR²⁰R²¹)ₙNR²²R²³, CO(CR²⁰R²¹)ₙOPO(OH)₂, SO(CR²⁰R²¹)ₙOPO(OH)₂, SO₂(CR²⁰R²¹)ₙOPO(OH)₂, CONR²⁰R²¹, SONR²⁰R²¹, SO₂NR²⁰R²¹, CNNR²⁰(CR²¹R²²)ₙCOOR²³, CNNR²⁰(CR²¹R²²)ₙOH, CNNR²⁰(CR²¹R²²)ₙNR²³R²⁴, CNNR²⁰(CR²¹R²²)ₙOPO(OH)₂, CONR²⁰(CR²¹R²²)ₙOH, SONR²⁰(CR²¹R²²)ₙOH, SO₂NR²⁰(CR²¹R²²)ₙOH, CONR²⁰(CR²¹R²²)ₙCOOR²³, SONR²⁰(CR²¹R²²)ₙCOOR²³, SO₂NR²⁰(CR²¹R²²)ₙCOOR²³CONR²⁰(CR²¹R²²)ₙNR²³R²⁴, SONR²⁰(CR²¹R²²)ₙNR²³R²⁴, SO₂NR²⁰(CR²¹R²²)ₙNR²³R²⁴, CONR²⁰(CR²¹R²²)ₙOPO(OH)₂, SONR²⁰(CR²¹R²²)ₙOPO(OH)₂, SO₂NR²⁰(CR²¹R²²)ₙOPO(OH)₂;
wherein n=0, 1, 2, 3, 4, 5 or 6;
and R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴ independently may represent H, C₁-C₆ alkyl, halogen, OH, (CR²⁵R²⁶)ₙCOOR²⁷, (CR²⁵R²⁶)ₙNR²⁷R²⁸, (CR²⁵R²⁶)ₙOH, (CR²⁵R²⁶)ₙOPO(OH)₂ and may together form a ring selected from morpholinyl, piperazinyl, 1-methylpiperazinyl, piperidinyl, pyrrolidinyl, imidazolyl, wherein R²⁵, R²⁶, R²⁷ and R²⁸ may represent H, C₁-C₆ alkyl, halogen and may together form a ring selected from morpholinyl, piperazinyl, 1-methylpiperazinyl, piperidinyl, pyrrolidinyl, imidazolyl; and any pharmaceutically acceptable salt thereof, the method including the step of applying the one or more fluorogenic probes to a sample and monitoring for the presence of at least one nitroreductase.

Preferably, the step of monitoring for the presence of nitroreductase includes the step of monitoring for the presence of a reduced fluorescent derivative of a compound of Formula I.

Preferably, the method further includes the step of quantifying the nitroreductase. More preferably, the step of quantifying the nitroreductase includes the step of quantifying the formation of the fluorescent derivative from the fluorescence emission intensity.

Preferably, in the compound of Formula I X is O or NH, Z is CH or NH and R³ is H.

Preferably the fluorogenic probe of Formula I is selected from
6-nitro-4(1H)-quinolinone,
1-methyl-6-nitro-4(1H)-quinolinone,
2-methyl-6-nitro-4(1H)-quinolinone,
N-[2-(dimethylamino)ethyl]-2-nitro-9-oxo-9,10-dihydro-4-acridinecarboxamide,
6-nitro-4(3H)-quinazolinone,
6-nitro-2-phenyl-4(3H)-quinazolinone,
methyl (6-nitro-4-oxo-1(4H)-quinolinyl)acetate,
N,N-dimethyl-3-[(7-nitro-4-quinolinyl)oxy]-1-propanamine,
N¹,N¹-dimethyl-N³-(7-nitro-4-quinolinyl)-1,3-propanediamine, and
N¹,N¹-dimethyl-N³-(5-nitro-4-quinazolinyl)-1,3-propanediamine and any pharmaceutically acceptable salt thereof.

In a fourth aspect there is provided a method for the fluorescent detection of nitroreductase activity using as one or more fluorogenic probes a class of nitro-substituted aromatic compounds of Formula II

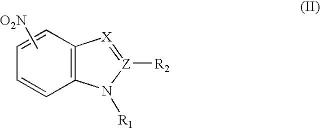

wherein X represents N, CH, O or S;
Z represents C or N;
wherein R¹ and R² if present, may independently represent H, C₁-C₆ alkyl, Ar, —(CH═CH)ₙAr, (CR³R⁴)ₙNR⁵R⁶, (CR³R⁴)ₙCOOR⁵, (CR³R⁴)ₙOH, (CR³R⁴)ₙOPO(OH)₂;
wherein n=0, 1, 2, 3, 4, 5 or 6;
wherein Ar may represent a substituted or unsubstituted phenyl, pyridyl, pyrimidinyl, thiazolyl, oxazoylyl, imidazolyl, furanyl, pyrrolyl, benzoxazolyl, benzthiazolyl, benzfuranyl, indolyl, indazolyl, benzimdazolyl, wherein each Ar is optionally substituted with one or more NO₂, CN, R³, OH, OR³, SH, SR³, halogen, CF₃, NH₂, NHR³, NR³R⁴, NHCOR³, NR³COR⁴, NHCOOR³, NR³COOR⁴, (CR³R⁴)ₙCOOH, (CR³R⁴)ₙCOOR⁵, (CR³R⁴)ₙCONH₂, (CR³R⁴)ₙCONHR⁵, (CR³R⁴)ₙCONR⁵R⁶, (CR³R⁴)ₙOH, (CR³R⁴)ₙOR⁵, (CR³R⁴)ₙOPO(OH)₂, COOH, COOR³, CONH₂, CONHR³, CONR³R⁴, COR³, SOR³, SO₂R³, SO₂NR³R⁴, SO₃H;
wherein R³, R⁴, R⁵ or R⁶ may independently represent H, C₁-C₆ alkyl, halogen, OH, (CR⁷R⁸)ₙNR⁹R¹⁰, (CR⁷R⁸)ₙOH, (CR⁷R⁸)ₙOPO(OH)₂ and may together form a ring selected from morpholinyl, piperazinyl, 1-methylpiperazinyl, piperidinyl, pyrrolidinyl, imidazolyl; wherein R⁷, R⁸, R⁹ and R¹⁰ may represent H or C₁-C₆ alkyl and may together form a ring selected from morpholinyl, piperazinyl, 1-methylpiperazinyl, piperidinyl, pyrrolidinyl, imidazolyl; and any pharmaceutically acceptable salts thereof the method including the step of applying the one or more fluorogenic probes to a sample and monitoring for the presence of at least nitroreductase.

Preferably, the step of monitoring for the presence of nitroreductase includes the step of monitoring for the presence of a reduced fluorescent derivative of a compound of Formula II.

Preferably, the method further includes the step of quantifying the nitroreductase. More preferably, the step of quantifying the nitroreductase includes the step of quantifying the formation of the fluorescent derivative from the fluorescence emission intensity.

Preferably, in the compound of Formula II X is N and Z is C, R¹=H and R²=Ar.

Preferably the compound of Formula II is selected from 5-nitro-2-phenyl-1H-benzimidazole and any pharmaceutically acceptable salt thereof.

In a fifth aspect there is provided a method for the fluorescent detection of nitroreductase activity using as one or more fluorogenic probes a class of nitro-substituted aromatic compounds of Formula III

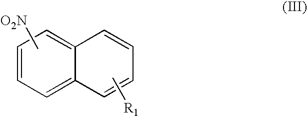

wherein R₁ may represent COR², SOR², SO₂R², CO(CR²R³)ₙOH, SO(CR²R³)ₙOH, SO₂(CR²R³)ₙOH, CO(CR²R³)ₙCOOR⁴, SO(CR²R³)ₙCOOR⁴, SO₂(CR²R³)ₙCOOR⁴, CO(CR²R³)ₙNR⁴R⁵, SO(CR²R³)ₙNR⁴R⁵, SO₂(CR²R³)ₙNR⁴R⁵, CO(CR²R³)ₙOPO(OH)₂, SO(CR²R³)ₙOPO(OH)₂, SO₂(CR²R³)ₙOPO(OH)₂, CONR²R³, SONR²R³, SO₂NR²R³, CNNR²(CR³R⁴)ₙCOOR⁵, CNNR²(CR³R⁴)ₙOH, CNNR²(CR³R⁴)ₙNR⁵R⁶, CNNR²(CR³R⁴)ₙOPO(OH)₂, CONR²(CR³R⁴)ₙOH, SONR²(CR³R⁴)ₙOH, SO₂NR²(CR³R⁴)ₙOH, CONR²(CR³R⁴)ₙCOOR⁵, SONR²(CR³R⁴)ₙCOOR⁵, SO₂NR²(CR³R⁴)ₙCOOR⁵, CONR²(CR³R⁴)ₙNR⁵R⁶, SONR²(CR³R⁴)ₙNR⁵R⁶, SO₂NR²(CR³R⁴)ₙNR⁵R⁶, CONR²(CR³R⁴)ₙOPO(OH)₂, SONR²(CR³R⁴)ₙOPO(OH)₂, SO₂NR²(CR³R⁴)ₙOPO(OH)₂;
wherein n=0, 1, 2, 3, 4, 5 or 6;
and R², R³, R⁴, R⁵, R⁶ independently may represent H, C₁-C₆ alkyl, halogen, OH, (CR⁷R⁸)ₙNR⁹R¹⁰, (CR⁷R⁸)ₙOH, (CR⁷R⁸)ₙOPO(OH)₂ and may together form a ring selected from morpholinyl, piperazinyl, 1-methylpiperazinyl, piperidinyl, pyrrolidinyl, imidazolyl; wherein R⁷, R⁸, R⁹ and R¹⁰ may represent H, C₁-C₆ alkyl, halogen and may together form a ring selected from morpholinyl, piperazinyl, 1-methylpiperazinyl, piperidinyl, pyrrolidinyl, imidazolyl; and any pharmaceutically acceptable salt thereof the method including the step of applying the one or more fluorogenic probes to a sample and monitoring for the presence of at least one nitroreductase.

Preferably, the step of monitoring for the presence of nitroreductase includes the step of monitoring for the presence of a reduced fluorescent derivative of a compound of Formula III.

Preferably, the method further includes the step of quantifying the nitroreductase. More preferably, the step of quantifying the nitroreductase includes the step of quantifying the formation of the fluorescent derivative from the fluorescence emission intensity.

Preferably, in the compound of Formula III R¹ is SO₂NR²(CR³CR⁴)ₙCOOR⁵, wherein R², R³, R⁴ and R⁵, may represent H, C₁-C₆ alkyl, halogen or OH and wherein n=0, 1, 2, 3, 4, 5 or 6.

Preferably the compound of Formula III is selected from
methyl 4-{[(5-nitro-1-naphthyl)sulfonyl]amino}butanoate,
methyl 4-{[(5-nitro-2-naphthyl)sulfonyl]amino}butanoate and
methyl 4-{[(8-nitro-2-naphthyl)sulfonyl]amino}butanoate and any pharmaceutically acceptable salt thereof.

In sixth aspect there is provided a class of nitroaromatic compounds of formula III′

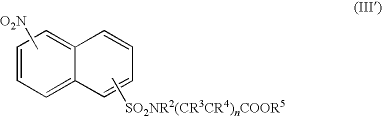

wherein R², R³, R⁴, R⁵ independently may represent H, C₁-C₆ alkyl, halogen, OH, (CR⁶R⁷)ₙNR⁸R⁹, (CR⁶R⁷)ₙOH, (CR⁶R⁷)ₙOPO(OH)₂ and may together form a ring selected from morpholinyl, piperazinyl, 1-methylpiperazinyl, piperidinyl, pyrrolidinyl, imidazolyl; wherein R⁶, R⁷, R⁸ and R⁹ may represent H, C₁-C₆ alkyl, halogen and may together form a ring selected from morpholinyl, piperazinyl, 1-methylpiperazinyl, piperidinyl, pyrrolidinyl, imidazolyl; and
wherein n=0, 1, 2, 3, 4, 5 or 6;
and any pharmaceutically acceptable salt thereof.

Preferably the compound of Formula III′ is selected from
methyl 4-{[(5-nitro-1-naphthyl)sulfonyl]amino}butanoate,
methyl 4-{[(5-nitro-2-naphthyl)sulfonyl]amino}butanoate and
methyl 4-{[(8-nitro-2-naphthyl)sulfonyl]amino}butanoate and any pharmaceutically acceptable salt thereof.

In a seventh aspect there is provided a method for the fluorescent detection of nitroreductase activity using as one or more fluorogenic probes a class of nitro-substituted aromatic compounds of Formula IV

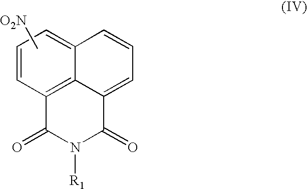

wherein R¹, may be selected from H, R², (CR²R³)ₙCOOH, (CR²R³)ₙCOOR⁴, (CR²R³)ₙCONH₂, (CR²R³)ₙCONHR⁴, (CR²R³)ₙCONR⁴R⁶, (CR²R³)ₙOH, (CR²R³)ₙOR⁴, (CR²R³)ₙOPO(OH)₂, (CR²R³)ₙNR⁴R⁶, (CR²R³)ₙ-morpholinyl, (CR²R³)ₙ-piperazinyl;
(CR²R³)ₙ-1-methylpiperazinyl; (CR²R³)ₙ-piperidinyl; (CR²R³)ₙ-pyrrolidinyl or (CR²R³)ₙ-imidazolyl; wherein n=0, 1, 2, 3, 4, 5 or 6;
and R², R³, R⁴, R⁵ independently may represent H, C₁-C₆ alkyl, halogen, OH, (CR⁶R⁷)ₙNR⁸R⁹, (CR⁶R⁷)ₙOH, (CR⁶R⁷)ₙOPO(OH)₂ and may together form a ring selected from morpholinyl, piperazinyl, 1-methylpiperazinyl, piperidinyl, pyrrolidinyl, imidazolyl; wherein R⁶, R⁷, R⁸ and R⁹ may represent H, C₁-C₆ alkyl, halogen and may together form a ring selected from morpholinyl, piperazinyl, 1-methylpiperazinyl, piperidinyl, pyrrolidinyl, imidazolyl; and any pharmaceutically acceptable salt thereof the method including the step of applying the one or more fluorogenic probes to a sample and monitoring for the presence of at least one nitroreductase.

Preferably, the step of monitoring for the presence of nitroreductase includes the step of monitoring for the presence of a reduced fluorescent derivative of a compound of Formula IV.

Preferably, the method further includes the step of quantifying the nitroreductase. More preferably, the step of quantifying the nitroreductase includes the step of quantifying the formation of the fluorescent derivative from the fluorescence emission intensity.

Preferably, in the compound of Formula IV R¹ is (CR²R³)ₙNR⁴R⁵.

Preferably the compound of Formula I is selected from 2-[4-(dimethylamino)butyl]-5-nitro-1H-benzo[de]isoquinoline-1,3(2H)-dione and any pharmaceutically acceptable salt thereof.

In a eighth aspect there is provided a method for the fluorescent detection of nitroreductase activity using as one or more fluorogenic probes a class of nitro-substituted aromatic compounds of Formula V

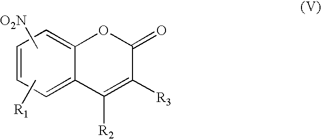

wherein R₁ may represent H, R⁴, COR⁴, SOR⁴, SO₂R⁴, CO(CR⁴R⁵)ₙOH, SO(CR⁴R⁵)ₙOH, SO₂(CR⁴R⁵)ₙOH, CO(CR⁴R⁵)ₙCOOR⁶, SO(CR⁴R⁵)ₙCOOR⁶, SO₂(CR⁴R⁵)ₙCOOR⁶, CO(CR⁴R⁵)ₙNR⁶R⁷, SO(CR⁴R⁵)ₙNR⁶R⁷, SO₂(CR⁴R⁵)ₙNR⁶R⁷, CO(CR⁴R⁵)ₙOPO(OH)₂, SO(CR⁴R⁵)ₙOPO(OH)₂, SO₂(CR⁴R⁵)ₙOPO(OH)₂, CONR⁴R⁵, SONR⁴R⁵, SO₂NR⁴R⁵, CNNR⁴(CR⁵R⁶)ₙCOOR⁷, CNNR⁴(CR⁵R⁶)ₙOH, CNNR⁴(CR⁵R⁶)ₙNR⁷R⁸, CNNR⁴(CR⁵R⁶)ₙOPO(OH)₂, CONR⁴(CR⁵R⁶)ₙOH, SONR⁴(CR⁵R⁶)ₙOH, SO₂NR⁴(CR⁵R⁶)ₙOH, CONR⁴(CR⁵R⁶)ₙCOOR⁷, SONR⁴(CR⁵R⁶)ₙCOOR⁷, SO₂NR⁴(CR⁵R⁶)ₙCOOR⁷, CONR⁴(CR⁵R⁶)ₙNR⁷R⁸, SONR⁴(CR⁵R⁶)ₙNR⁷R⁸, SO₂NR⁴(CR⁵R⁶)ₙNR⁷R⁸, CONR⁴(CR⁵R⁶)ₙOPO(OH)₂, SONR⁴(CR⁵R⁶)ₙOPO(OH)₂, SO₂NR⁴(CR⁵R⁶)ₙOPO(OH)₂;
R² and R³ may independently represent H, R⁹, (CR⁹R¹⁰)ₙCOOH, (CR⁹R¹⁰)ₙCOOR¹¹, (CR⁹R¹⁰)ₙCONH₂, (CR⁹R¹⁰)ₙCONHR¹¹, (CR⁹R¹⁰)ₙCONR¹¹R¹², (CR⁹R¹⁰)ₙOH, (CR⁹R¹⁰)ₙOR¹¹, (CR⁹R¹⁰)ₙOPO(OH)₂, COOH, COOR⁹, CONH₂, CONHR⁹, CONR⁹R¹⁰, COR⁹, CN, SOR⁹, SO₂R⁹, SO₂NR⁹R¹⁰, or R² and R³ may together form a fused aromatic ring optionally substituted at one or more of the available carbons with a C₁-C₆ alkyl, halogen, SO₃H or CN;
wherein n=0, 1, 2, 3, 4, 5 or 6;
and R⁴, R⁵, R⁶, R⁷, R⁸, R⁹, R¹⁰, R¹¹, R¹² independently may represent H, C₁-C₆ alkyl, halogen, OH, (CR¹³R¹⁴)ₙNR¹⁵R¹⁶, (CR¹³R¹⁴)ₙOH, (CR¹³R¹⁴)ₙOPO(OH)₂ and may together form a ring selected from morpholinyl, piperazinyl, 1-methylpiperazinyl, piperidinyl, pyrrolidinyl, imidazolyl; wherein R¹³, R¹⁴, R¹⁵ and R¹⁶ may represent H, C₁-C₆ alkyl, halogen and may together form a ring selected from morpholinyl, piperazinyl, 1-methylpiperazinyl, piperidinyl, pyrrolidinyl, imidazolyl; and any pharmaceutically acceptable salt thereof the method including the step of applying the one or more fluorogenic probes to a sample and monitoring for the presence of at least one nitroreductase.

Preferably, the step of monitoring for the presence of nitroreductase includes the step of monitoring for the presence of a reduced fluorescent derivative of a compound of Formula V.

Preferably, the method further includes the step of quantifying the nitroreductase. More preferably, the step of quantifying the nitroreductase includes the step of quantifying the formation of the fluorescent derivative from the fluorescence emission intensity.

Preferably, in the compound of Formula V R² and R³ together represent Ar.

Preferably the compound of Formula V is selected from 3-nitro-6H-benzo[c]chromen-6-one and any pharmaceutically acceptable salt thereof.

Preferably in the method aspects defined above the one or more nitroreductase is a human oxidoreductase selected from known human enzymes classified as EC 1 in the EC number classification of enzymes. Oxidoreductases are classified into 22 subclasses of which 6 have known nitroreductase activities:

EC 1.1 includes oxidoreductases that act on the CH—OH group of donors

e.g. Aldose reductase [ALDR1; E.C.1.1.1.21];
e.g. Aldehyde reductase [AKR1B10; E.C.1.1.1.2].
EC 1.2 includes oxidoreductases that act on the aldehyde or oxo group of donors
EC 1.3 includes oxidoreductases that act on the CH—CH group of donors
EC 1.4 includes oxidoreductases that act on the CH—NH₂ group of donors
EC 1.5 includes oxidoreductases that act on CH—NH group of donors
EC 1.6 includes oxidoreductases that act on NADH or NADPH
e.g. DT-diaphorase [NQO1; E.C.1.6.99.2];
e.g. Cytochrome P450-reductase [CYPOR; E.C.1.6.2.4];
e.g. Cytochrome B5 reductase [DIA1; E.C.1.6.2.2];
EC 1.7 includes oxidoreductases that act on other nitrogenous compounds as donors
EC 1.8 includes oxidoreductases that act on a sulfur group of donors
e.g. Thioredoxin-disulfide reductase [TXNRD; E.C.1.8.1.9];
EC 1.9 includes oxidoreductases that act on a heme group of donors
EC 1.10 includes oxidoreductases that act on diphenols and related substances as donors
EC 1.11 includes oxidoreductases that act on peroxide as an acceptor (peroxidases)
EC 1.12 includes oxidoreductases that act on hydrogen as donors
EC 1.13 includes oxidoreductases that act on single donors with incorporation of molecular oxygen (oxygenases)
EC 1.14 includes oxidoreductases that act on paired donors with incorporation of molecular oxygen
e.g. Inducible nitric oxide synthase [NOS2A; E.C.1.14.13.39];
EC 1.15 includes oxidoreductases that act on superoxide radicals as acceptors
EC 1.16 includes oxidoreductases that oxidize metal ions;
e.g. Methionine synthase reductase [MTRR; E.C.1.16.1.8];
EC 1.17 includes oxidoreductases that act on CH or CH₂ groups;
e.g. Xanthine oxidase [XO; E.C.1.17.3.2];
e.g. Xanthine dehydrogenase [XDH; E.C.1.17.1.4];
EC 1.18 includes oxidoreductases that act on iron-sulfur proteins as donors;
e.g. Adrenodoxin oxidoreductase [FDXR; E.C.1.18.1.2]
EC 1.19 includes oxidoreductases that act on reduced flavodoxin as a donor
EC 1.21 includes oxidoreductases that act on X—H and Y—H to form an X—Y bond
EC 1.97 includes other oxidoreductases

In the alternative, the one or more nitroreductase is a microbial or fungal nitroreductase selected from type I nitroflavin reductase NfsA-like and NfsB-like superfamilies or the NQO1-like and YieF-like nitroreductase enzymes, or any putative nitroreductase gene showing evidence of significant sequence homology thereof.

In the ninth aspect of the invention there is provided a method for identifying the presence of cellular hypoxia by contacting in a first step an effective amount of a compound of Formula I to V as defined above to the one or more cellular samples, and in a second step monitoring for the formation of a fluorescent derivative arising from the reduction of the nitro group of the compound of Formula I to V by one or more nitroreductase present in the cellular sample.

Preferably the one or more nitroreductase is a human nitroreductase selected from

### DT-diaphorase [NQO1; E.C.1.6.99.2];

Cytochrome P450-reductase [CYPOR; E.C.1.6.2.4];
Inducible nitric oxide synthase [NOS2A; E.C.1.14.13.39];
Cytochrome B5 reductase [DIA1; E.C.1.6.2.2];
Xanthine oxidase [XO; E.C.1.17.3.2];
Xanthine dehydrogenase [XDH; E.C.1.17.1.4];
Adrenodoxin oxidoreductase [FDXR; E.C.1.18.1.2];
Methionine synthase reductase [MTRR; E.C.1.16.1.8];
Aldose reductase [ALDR1; E.C.1.1.1.21]; and
Aldehyde reductase [AKR1B10; E.C.1.1.1.2]
Thioredoxin reductase [TXNRD; E.C.1.8.1.9]

Preferably the method further includes the analytical step of quantifying the formation of the fluorescent derivative from the fluorescence emission intensity.

In a tenth aspect of the invention, there is provided an assay for the detection of nitroreductase including the steps of:
(i) contacting an effective amount of a plurality of fluorogenic probes with a sample;
(ii) monitoring for the formation of fluorescent derivatives.

Preferably, the sample may be added to a common test environment containing a plurality of fluorogenic probes.

In an eleventh aspect of the invention, there is provided an assay for the detection of nitroreductase including the steps of:
(iii) contacting an effective amount of at least one compounds of formula I-V as defined in the second aspect, with a sample;
(iv) monitoring for the formation of fluorescent derivatives.

In a twelfth aspect of the invention, there is provided an assay comprising at least one test environment containing a plurality of fluorogenic probes, wherein a sample may be added and the test environment monitored for the formation of fluorescent derivatives.

Preferably, the fluorogenic probes are selected from compounds of formula I-V as defined in the second aspect.

Preferably the test environment is compatible with sustained cell viability, permitting real time multiple analyses with synchronous detection.

In a thirteenth aspect of the invention, there is provided a compound of formula I-V as defined in the second aspect, wherein one or more nitro substituents is replaced by an amine or hydroxylamine moiety.

Further aspects of the present invention will become apparent from the following Figures and Examples which are given by way of example only:

### BRIEF DESCRIPTION OF DRAWINGS

FIG. 1 shows the structural representations of representative nitro-substituted aromatic compounds 1 to 16 of the present invention.

FIG. 2 shows representative fluorescent amino-substituted aromatic compounds 17 to 23 of the present invention.

FIG. 3 shows the fluorescent intensity observed for compounds 1 to 15 when reduced in the presence of E. coli nitroreductase (nfsB).

FIG. 4 shows the rate of fluorescence signal generation for compound 2 when reduced in the presence of E. coli nitroreductase (nfsB).

FIG. 5 shows the fluorescent intensity observed for compounds 1 to 15 when reduced in the presence of human aerobic reductase NAD(P)H dehydrogenase quinone 1 (NQO1).

FIG. 6 shows the rate of fluorescence signal generation for compound 1 when reduced in the presence of human aerobic reductase NAD(P)H dehydrogenase quinone 1 (NQO1).

FIG. 7 shows the shows the fluorescent intensity observed for compounds 1 to 15 when reduced in the presence of the human anaerobic reductase NADPH Cytochrome P450 reductase (CYPOR).

FIG. 8 shows the fluorescent intensity of compounds 1 and 13 when reduced in the presence of E. coli nitroreductase B (nfsB) expressing cells co-cultured in the presence of non-expressing cells. Cells were washed and media was replaced after 1 hour with fluorescence monitored over a 4 hour time frame.

FIG. 9 shows the fluorescent intensity of compounds 4, 13, 14, and 15 when reduced in the presence of E. coli nitroreductase (nfsB) expressing cells (B) or non-expressing cells (A) for 1.5 hr and imaged 6 hours after cells were washed free and fresh media was replaced. (NB The image has been rendered monochromatic for the purposes of publication quality.)

FIG. 10 shows the superiority of compound 2 relative to methyl 7-nitrocoumarin carboxylate (methyl 7-nitro-2-oxo-2H-chromene-3-carboxylate) as described in US20020031795A1 as a nitroreductase fluorescent reporter.

FIG. 11 shows the superior aqueous stability of compound 1 and 2 compared with the disclosed compounds methyl-7-nitrocoumarin carboxylate and 7-nitrocoumarin-3-carboxylic acid as described in US20020031795A1 and Letters in Applied Microbiology, (33) 403-8, 2001.

FIG. 12 shows the superior aqueous stability under aerobic or anaerobic conditions of compounds 1 to 15 compared with the disclosed compounds methyl-7-nitrocoumarin carboxylate, 7-nitrocoumarin-3-carboxylic acid and 6-chloro-9-nitro-5H-benzo[a]phenoxazin-5-one (Molecular Probes Handbook, 10^{th} Edition, page 535).

FIG. 13 shows 3 fluorescent amine reporter molecules of non-overlapping excitation/emission spectra suitable for multiplexed reporter gene applications.

FIG. 14 shows the multiplex use of compounds 4 and 16 to identify concurrently two nitroreductase expressing cell populations in a common environment.

FIG. 15 shows the multiplex use of compounds 11 and 13 to identify concurrently two nitroreductase expressing cell populations in a common environment.

### DETAILED DESCRIPTION OF THE INVENTION

The invention broadly relates to a method for the detection of nitroreductase activity using at least one fluorogenic probe. More specifically, the invention relates to a method that may be adapted to detect and/or identify a plurality of nitroreductases sequentially or simultaneously in a common test environment. The ability to use a common environment for multiple determinations leads to advantages in assay systems for detection and/or diagnosis. A single detection method can be used (e.g. fluorescence emission) without the need to quench between readings, permitting time-dependent monitoring using such noninvasive detection methods. When multiplexed on sub-cellular imaging systems including; microscopes, sub-cellular imagers and plate readers, relationships between multiple reporter gene cell populations, or single cell populations harboring multiple nitroreductase reporter genes can be quantified as a function of time. This avoids the pitfalls of cell lysis, synchronizes detection, and allows for specific and direct comparison of two or more promoter-regulated nitroreductase activities in a multiplex format.

The method utilises one or more fluorogenic probes, which may be reduced by the action of one or more nitroreductase(s), resulting in one or more strongly fluorescent molecules. Wherein the fluorescent output may be detected simultaneously or sequentially in a common test environment.

The fluorogenic probes of the invention are readily available and are stable in their non-fluorescent nitro form and as the fluorescent reduced amine or hydroxylamine derivatives. While multiple probes may co-exist in the fluorescent and non-fluorescent forms in a common test environment, the presence of individual fluorescent derivatives may be quickly and easily detected independently. The fluorescent derivatives may be independently detected either sequentially or simultaneously by monitoring their often characteristic fluorescence emission.

The inventors have employed singleton synthesis and substructure screening of in-house chemical libraries to collate a Fluorogenic Substrate Library (FSL) including of a range of nitro-substituted aromatic compounds that are likely to be fluorescent upon bioreduction. High-throughput fluorogenic cell-based screening assays have been developed and several fluorogenic probes have been identified for specific nitroreductases.

The nitroreductases that can be detected by this technology may be of microbial or human origin, for example the Escherichia coli oxygen-insensitive minor nitroreductase (NTR) [nfsB], or human DT-diaphorase (DTD) [NQO1; E.C.1.6.99.2] and human cytochrome P450-reductase (P450R) [CYPOR; E.C.1.6.2.4]. Other human nitroreductases may include

Inducible nitric oxide synthase [NOS2A; E.C.1.14.13.39], Cytochrome B5 reductase [DIA1; E.C.1.6.2.2]; Xanthine oxidase [XO; E.C.1.17.3.2]; Xanthine dehydrogenase [XDH; E.C.1.17.1.4]; Adrenodoxin oxidoreductase [FDXR; E.C.1.18.1.2]; Methionine synthase reductase [MTRR; E.C.1.16.1.8]; Aldose reductase [ALDR1; E.C.1.1.1.21]; Aldehyde reductase [AKR1B10; E.C.1.1.1.2] and Thioredoxin reductase [TXNRD; E.C.1.8.1.9] or any appropriate human oxidoreductase from enzyme class EC 1.1.

Nitro reductases such as NTR and DTD have been shown to catalyse oxygen-insensitive two electron reduction of a nitro (NO₂) group to a hydroxylamine (NHOH) group (the four electron reduction product) which may be subsequently reduced to an amine group (the six electron reduction product), while nitroreductases such as P450R catalyse reduction that proceeds via an oxygen-sensitive one electron intermediate as shown in Scheme 1. In the presence of oxygen this one electron intermediate is back-oxidised to regenerate the starting material. In the absence of oxygen (hypoxia) further reduction to a hydroxylamine and amine can occur.

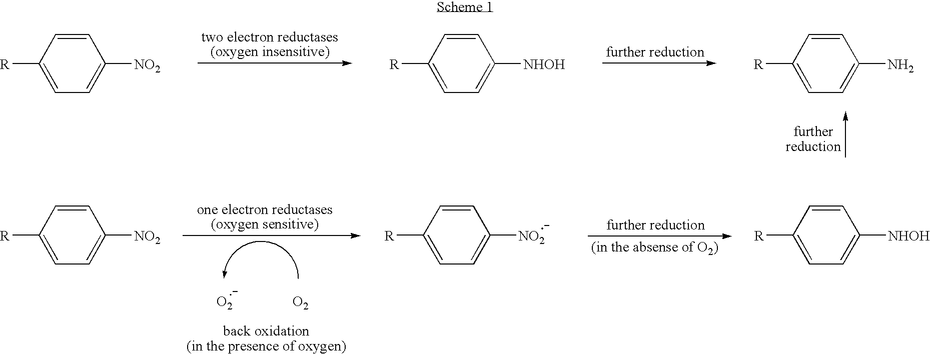

In the present invention the nitro containing molecules (substrates) of interest are non-fluorescent dyes which upon metabolism by a nitroreductase yield stable fluorescent products (the hydroxylamine and amine containing compounds) that emit light upon excitation over a broad range of the spectrum that is proportional to their concentrations. Therefore, metabolic conversion of substrates yields products that are strongly fluorescent, reporting the presence of nitroreductase activity.

The stable fluorescent derivatives may be excited using light from the UV/visible spectrum and the fluorescent emission determined using any instrument adapted to detect and quantify light emissions, for example. a UV/vis spectrometer. Compounds of the invention will generally also emit in the UV/visible/IR range (200-800 nm).

### Applications:

### Use and Detection of Microbial or Fungal Nitroreductases in Enzyme-Reporter Assays

Any non-ubiquitous enzyme which does not occur naturally may be inserted into a cell of interest in such a way that expression of the enzyme is linked to the expression of a cellular gene of interest. For example, it may be placed under the control of an appropriate transcriptional or post-transcriptional control sequence. A nitroreductase of microbial, fungal or mammalian origin utilised in this context is defined as a reporter gene.

The catalytic generation of a fluorescent signal from a non-fluorescent substrate correlates with the expression of the reporter gene, thus providing a quantitative and/or spatial measure of the activity of the regulatory sequence and expression of a gene of interest. In certain utilities, the fluorescent product may be entrapped within the cell of origin thereby identifying individual cells or tissue regions expressing the reporter gene at the time of compound exposure. Uses of entrapped and freely-diffusing probes can include high-throughput cell based screening assays for compound discovery or identification of regional reporter gene expression within tissue regions of interest. This may include identification of nitroreductase delivered by exogenous vector systems, for example gene therapy, or expressed from tissue specific promoters, for example transgenic animals. Probe use may include identification of cells for subsequent nitroreductase-mediated ablation therapy.

The reporter enzyme may be coupled to an assay component of any binding assay such as an antibody/antigen in an immunoassay or a hormone/receptor in an affinity assay or a nucleic acid molecule in a nucleic acid hybridization assay (DNA/DNA, DNA/RNA, DNA/protein) or biotin/streptavidin or lectin/glycoprotein. The conversion of a non-fluorescent substrate to a fluorescent product provides a measure of bound nitroreductase activity and correlates with the amount of analyte in the assay.

### Multiplex Detection of Nitroreductase Enzyme Activity

The various compounds of the invention incorporate a variety of chromophores and as such can be utilised to determine the presence of at least one nitroreductase in a single test environment. The reduced derivatives of the compounds of the invention fluoresce at characteristic wavelengths. Subsequently, the detection of a characteristic emission signal indicates the presence of a particular reduced substrate. As many of the reduced substrates have unique characteristic emission signals, more than one reduced substrate can be detected in a single/common test environment.

Specific compounds of the invention may function as indicative probes for specific nitroreductases. Therefore, a specific nitroreductase may be identified on detection of one or more fluorescent probes. Furthermore, as more than one fluorescent probe can be detected in a common test environment, the method can be applied to identify multiple nitroreductase enzymes in a common test environment.

The fluorescent probes can be detected sequentially or simultaneously in the common test environment, as desired by the user. This provides process advantages over other similar assay type systems, which require separate reagents and/or detection methods for each probe used. The ability to obtain multiple results from a single test will allow much faster screening of samples resulting in improved efficiency in detection and/or diagnostic methods. No requirement is imposed for each reporter activity to be assayed separately, generally employing different chemistry and detection methods, with sequential measurements and iterative chemistry steps or sample separation.

In a preferred embodiment of the invention, the emission spectra of the florescent probes used in the multiplex environment will be sufficiently discrete to allow detection of the individual fluorescent derivatives.

Persons skilled in the art will appreciate the type of common test environment assay that may be used to perform the invention. Test environments can include high throughput small molecule of biological molecule screening platforms designed to establish the differential biological effects on one cell population over another, or effects on a specific signal transduction pathway relative the another in order to aid in the identification of agents that are active for a given utility. This can include, but is not limited to the use of differential promoter assay to identify modulators of certain signal transduction pathways and mixed cell populations where an intended effect upon a subpopulation is desired. Screening can be conducted in separate cell populations that can be subsequently mixed in a single test environment or multiple promoter activities within a single cell population. Other platforms can include single cell fluorescent microscopy with high content image analysis for high throughput applications, including signal ratio calculations of multiplex signals to provide additional information relating to the differential activity of reporter gene nitroreductases in a common test environment. Those skilled in the art of cellular physiology and microscopy will appreciate the many other applications of enzyme generated fluorescent signal detection, including confocal microscopic detection of cell populations to monitor intracellular processes such as protein trafficking with the aid of split excitation and laser photobleaching.

By way of example, a sample can be applied to an assay test environment (eg an assay well) containing a plurality of fluorogenic probes. Light from the UV/visible spectrum can be used to excite any reduced derivatives in the test environment, which may fluoresce at a characteristic wavelength, thus indicating the presence of specific nitroreductase enzyme(s).

### Detection of Microbial Nitroreductase Enzyme Activity

Compounds disclosed in this invention may be used in the detection and/or diagnostic tests for microorganisms. Nitroreductase activity is common, being found in the majority of organisms including obligate aerobic and anaerobic bacteria, fungi and eukaryotic parasites. Conversion of a non-fluorescent substrate to a fluorescent product provides a universal test for the presence of microorganisms in samples or cultures. Uses may include, but are not limited to, bioremediation, sterility tests, antibiotic susceptibility and quantification of organisms present in any sample.

The invention may be employed to demonstrate the presence of nitroreductase activity in any test sample containing one or more microorganisms of commercial value (e.g. food product, soil sample, aqueous sample) or medical interest (e.g. body fluids).

### Detection of Mammalian Nitroreductase Enzyme Activity

Compounds disclosed in this invention may be used in the detection and/or diagnostic tests for human nitroreductase activity. Under aerobic conditions certain obligate two-electron reductases, for example NAD(P)H dehydrogenase quinone 1 (DT-Diaphorase; NQO1, E.C.1.6.99.2), can be detected in living tissue samples or preparations there of. Alternately, in the absence of oxygen (hypoxia) certain one-electron reductases, for example NADPH cytochrome P450 reductase (CYPOR, E.C.1.6.2.4), can be detected in living tissue samples or preparations there of. Alternatively, compounds may be employed to detect the total (composite) reductive activity of ubiquitous one-electron reductase activities. This has utility for predicting total reductive catalytic capacity of living tissue samples or preparations there of and may be of value in predicting reductive metabolism of therapeutic agents, for example hypoxic cytotoxins.

In all cases the conversion of a non-fluorescent substrate to a fluorescent product provides a measure of nitroreductase activity of interest and correlates with catalytic activity in the sample. In certain utilities, the fluorescent product may be entrapped within the cell of origin thereby identifying it as expressing the reductase of interest at the time of compound exposure. Signal retention may correlate with amplitude of enzyme catalysis providing a measure of the heterogeneity within a cell, tissue or analyte sample series.

### Detection of Cellular Hypoxia

Compounds disclosed in this invention may be used in the detection and/or diagnostic tests for tissue hypoxia (<1% O₂). Conversion of a non-fluorescent substrate to a fluorescent product by ubiquitous one-electron reductases, which occurs selectively in the absence of oxygen, provides a test for the relative absence of oxygen in a specific cell population or tissue region. In certain utilities, the fluorescent product may be entrapped within the cell of origin thereby identifying it as hypoxic at the time of compound exposure.

Additionally, the conversion of a non-fluorescent substrate to a fluorescent product by an oxygen-inhibited reductase can provide a measure of hypoxia in any test system of interest. The generation of fluorescence signal correlates with the concentration of oxygen in the sample.

### EXAMPLES

The present invention will now be described in more detail by referring to the following examples, but is not deemed to be limited thereto.

### Example 1

### Method of Detecting E. Coli Nitroreductase (nfsB) Reporter Gene Activity in Intact Cells

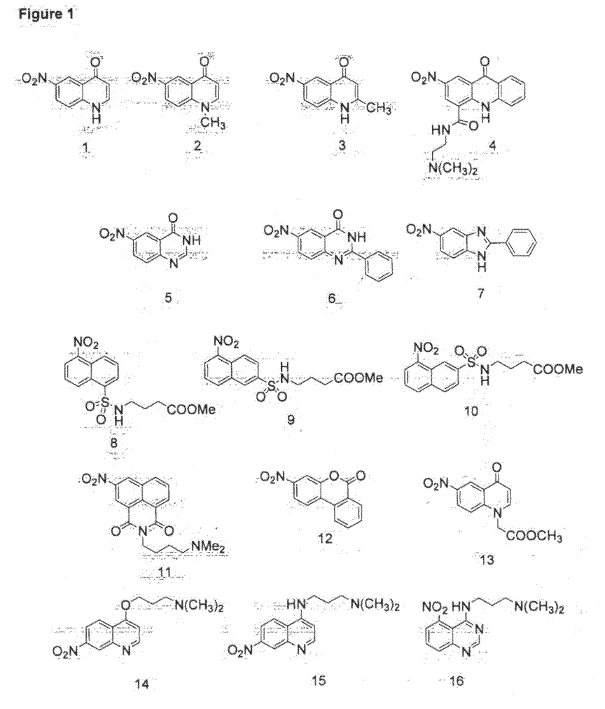

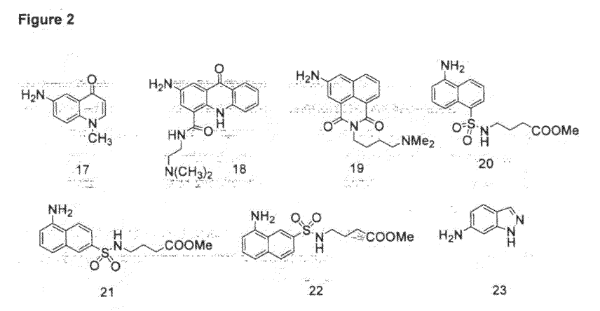

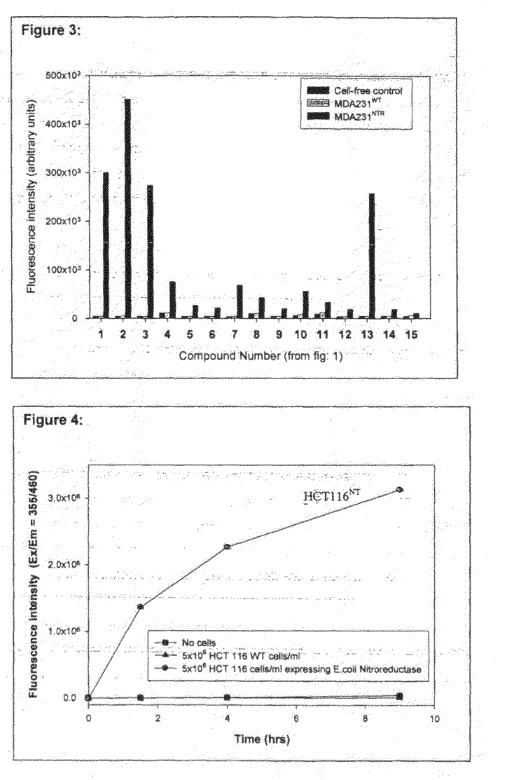

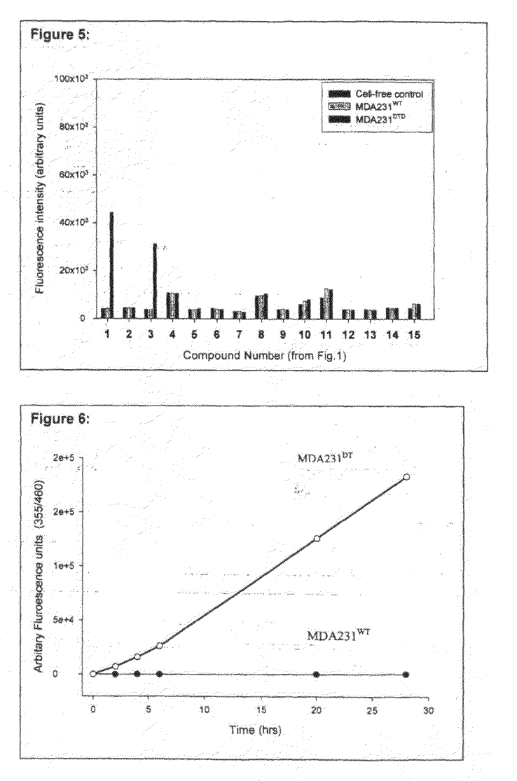

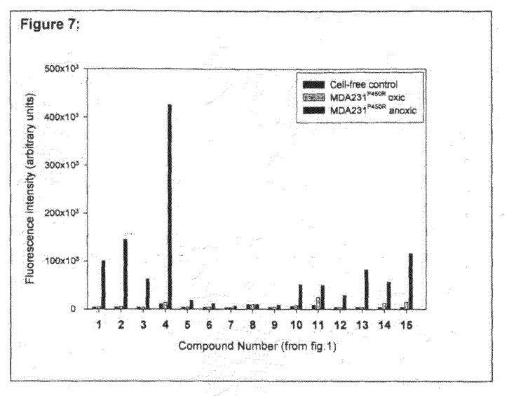

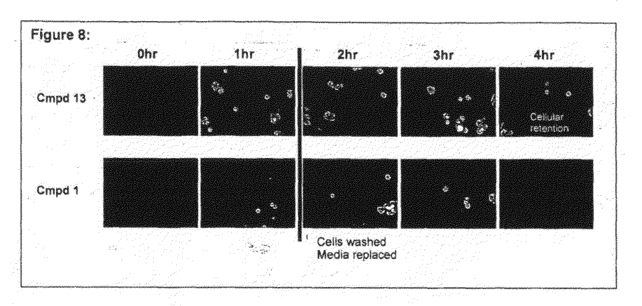

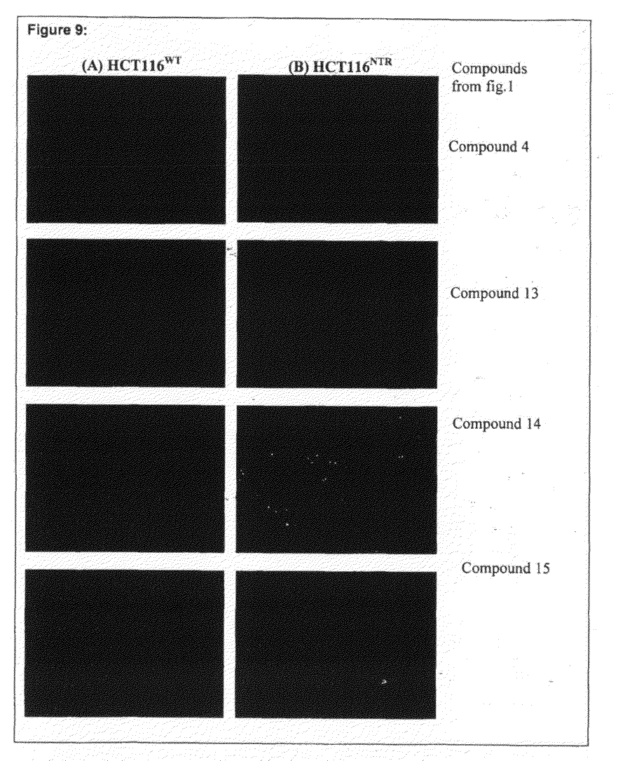

The human breast cancer cell line (MDA231^{WT}) and a clonal derivative (MDA231^{NTR}) engineered to express the reporter gene E. coli nitroreductase (nfsB) under the control of a constitutive promoter were seeded into 96-well plates at a density of 1×10⁵ cells/well. When samples were equilibrated to 37° C., 5% CO₂ for 2 hr and compounds 1 to 15 (FIG. 1) were added to a final concentration of 100 μM for 4 hr. Test groups were cell-free culture media alone (control), MDA231^{WT} and MDA231^{NTR}. The fluorescence signal was monitored at an excitation wavelength of 355 nm and emission wavelength of 460 nm (355/460) except for compounds 4 and 11 that were monitored at 405/585 and compounds 8 and 10 that were monitored at 355/585 and 355/535 respectively (FIG. 1). No fluorescence was observed in either the cell-free control or parental MDA231^{WT} containing cultures. Compounds 1-15 inclusive gave rise to a fluorescent signal specifically in the presence of E. coli nitroreductase (nfsB) expression.

In a further exemplification, the human colorectal cancer cell line (HCT116^{WT}) and a clonal derivative (HCT116^{NTR}) engineered to express the reporter gene E. coli nitroreductase (nfsB) under the control of a constitutive promoter were suspended in stirred culture media at a density of 5×10⁶ cells/ml. When samples were equilibrated to 37° C., 5% CO₂, compound (2) (1-methyl-6-nitro-4(1H)-quinolinone) was added to a final concentration of 300 μM. Test groups were culture media alone (control), HCT116^{WT} and HCT116^{NTR}. The rate of fluorescence signal generation at 355/460 was monitored as a function of time (FIG. 4). HCT116^{NTR} cells rapidly reduced compound (2) a process that approached completion by 9 hrs. No detectable fluorescence was observed in either the control or parental HCT116^{WT} containing cultures.

### Example 2

### Method of Detecting Human Aerobic Nitroreductase Activity

The human breast cancer cell line (MDA231^{WT}) and a clonal derivative (MDA231^{DTD}) engineered to express the human aerobic reductase, NAD(P)H dehydrogenase quinone 1 (DT-diaphorase; NQO1) under the control of a constitutive promoter were seeded into 96-well plates at a density of 1×10⁵ cells/well. When samples were equilibrated to 37° C., 5% CO₂ for 2 hr and compounds 1 to 15 were added to a final concentration of 100 μM for 4 hr. Test groups were cell-free culture media alone (control), MDA231^{WT} and MDA231^{DTD}. The fluorescence signal was monitored at 355/460 except for compounds 4 and 11 that were monitored at 405/585 and compounds 8 and 10 that were monitored at 355/585 and 355/535 respectively (FIG. 5). No detectable fluorescence was observed in either the control or parental MDA231^{WT} containing cultures. Compounds 1 and 3 gave rise to a fluorescent signal specifically in the presence of human NQO1 expression.

In a further exemplification, the human breast cancer cell line (MDA231^{WT}) and a clonal derivative (MDA231^{DTD}) engineered to express the human NQO1 gene (DT-diaphorase) under the control of a constitutive promoter were seeded into 96-well tissue culture plates at 1×10⁵ cells/well. Samples were equilibrated to 37° C., 5% CO₂, compound (1) (6-nitro-4(1H)-quinolinone) was added to a final concentration of 300 μM. Test groups were culture media alone (control), MDA231^{WT} cells and MDA231^{DTD} cells. The rate of fluorescence signal generation at 355/460 was monitored as a function of time (FIG. 6). MDA231^{DTD} cells reduced compound 1 and 3 (see FIG. 5) to generate a fluorescent signal. No detectable fluorescence was observed in either the wells containing compound 1 alone (control) or parental MDA231^{WT}.

### Example 3

### Method of Detecting Human Nitroreductase Activity Under Anoxia

A clonal derivative of the human breast cancer cell line (MDA231^{P450R}), engineered to overexpress the human anaerobic reductase, NADPH cytochrome P450 reductase (CYPOR) under the control of a constitutive promoter were seeded into 96-well plates at a density of 1×10⁵ cells/well. When samples were equilibrated to 37° C., 5% CO₂ for 2 hr under 95% N₂ and compounds 1 to 15 were added to a final concentration of 100 μM for 4 hr. Test groups were cell-free culture media alone (control), MDA231^{P450R} under normoxic (air) and anoxic (N₂) conditions. The fluorescence signal was monitored at 355/460 except for compounds 4 and 11 that were monitored at 405/585 and compounds 8 and 10 that were monitored at 355/585 and 355/535 respectively (FIG. 7). No detectable fluorescence was observed in either the control or aerobic MDA231^{P450R} containing cultures. Compounds 1-5 and 10-15 gave rise to a fluorescent signal specifically in the presence of human cytochrome P450 reductase expression when oxygen was absent.

### Cellular Entrapment:

A particularly attractive property of a number of fluorogenic probes described herein is that of cellular entrapment of the fluorescent reporter molecule produced upon nitroreductase activity.

These compounds comprise of at least one NO₂ group and at least one of the groups R¹, R², R³, R⁴, R⁵ of the molecule of formula I, R¹, R² of formula II, R¹ of formula III, R², R³, R⁴, R⁵ of formula III′, R¹ of formula IV, R¹, R², R³ of formula V that provides for cell membrane permeabilising properties. Membrane permeant compounds can generally be provided by masking hydrophilic groups. After entry into the cell the masking group can be designed to be cleaved to produce a hydrophilic fluorogenic substrate that provides a cell entrapped fluorescent report in the presence of reductase activity. Alternately, compounds comprising of at least one NO₂ group and at least one of the groups R¹, R², R³, R⁴, R⁵ of the molecule of formula I, R¹, R² of formula II, R¹ of formula III, R², R³, R⁴, R⁵ of formula III′, R¹ of formula IV, R¹, R², R³ of formula V that provides for increased DNA affinity can provide a nuclear localised cell entrapped fluorescent report in the presence of nitroreductase activity.

### Example 4

### Entrapment of Fluorescent Products within Nitroreductase Expressing Cells

The human colorectal cancer cell line (HCT116^{WT}) and a clonal derivative (HCT116^{NTR}) engineered to express the reporter gene E. coli nitroreductase (nfsB) under the control of a constitutive promoter were seeded onto glass coverslips at an equal density (50:50). Cells were equilibrated to 37° C., 5% CO₂, and compound 1 and 13 was added to a final concentration of 100 μM for 1 hr. Cells were washed free of compound after 1 hour and fluorescence was monitored as a function of time (FIG. 8). By 4 hours, cells were no longer fluorescent following exposure to compound 1, whereas compound 13, bearing an ester group subject to intracellular hydrolysis, was still retained within the nitroreductase expressing (but not co-cultured parental) cells. This demonstrates that durable and specific retention of fluorescence is possible with nitroreductase positive cells even when co-cultured in the presence of non-expressing cells.

In further screening (FIG. 9), the human colorectal cancer cell line (HCT116^{WT}) and a clonal derivative (HCT116^{NTR}) engineered to express the reporter gene E. coli nitroreductase (nfsB) under the control of a constitutive promoter were seeded separately into a 96-well glass plate. Cells were equilibrated to 37° C., 5% CO₂, and compounds were added to a final concentration of 10 μM for 1.5 hr. Cells were washed free of compound and fluorescence was monitored 6 hours post-exposure. Compounds 4, 13, 14 and 15 are identified as possessing cellular retention properties, with fluorescence being observed within the nitroreductase expressing (B) but not parental (A) HCT116 cells. This demonstrates that durable and specific retention of fluorescence is possible with nitroreductase positive cells. Parental non-expressing cells failed to provide a signal. The image has been rendered monochromatic for the purposes of publication quality.

### Example 5

### Superior Nitroreductase Specificity of Compound 2 Over 7-Nitrocoumarins

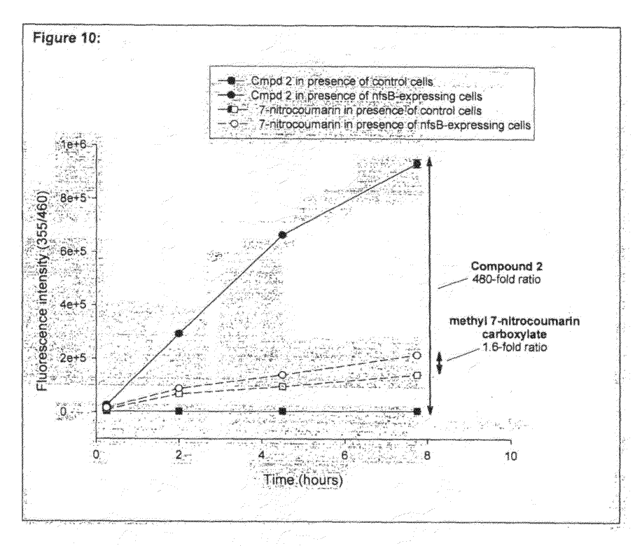

The human breast cancer cell line (MDA231^{WT}) and a clonal derivative (MDA231^{NTR}) engineered to overexpress the E. coli nitroreductase (nfsB) under the control of a constitutive promoter were seeded into 96-well plates at a density of 2×10⁴ cells/well. When samples were equilibrated to 37° C., 95% N₂, 5% CO₂ for 2 hr and compound 2 or methyl 7-nitrocoumarin carboxylate (methyl 7-nitro-2-oxo-2H-chromene-3-carboxylate) were added to a final concentration of 300 μM. The fluorescence signal was monitored at 355/460 over a 7.5 hour time frame (FIG. 10). For compound 2 no detectable fluorescence (above background) was observed in the control cell line MDA231^{WT} containing cultures, whereas a robust fluorescent signal was observed in the nitroreductase-expressing cell line MDA231^{NTR} giving rise to a 480-fold differential signal at 7.5 hours. In contrast, the methyl 7-nitrocoumarin carboxylate compound generated measurable fluorescence in the control cell line MDA231^{WT} containing cultures, which was only elevated 1.6-fold in the nitroreductase-expressing cell line MDA231^{NTR}. Therefore compound 2 is demonstrably superior to methyl 7-nitrocoumarin carboxylate as disclosed in US20020031795A1.

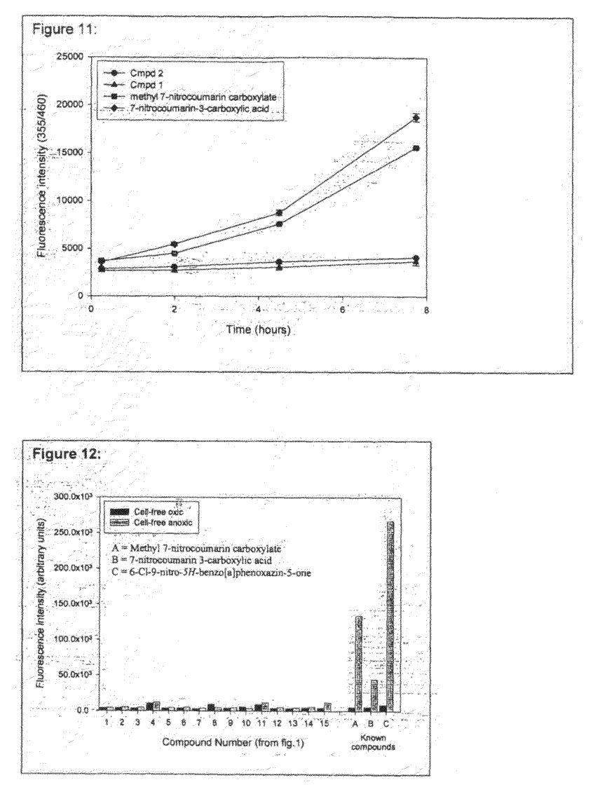

In a further experiment, the aqueous stability of compounds 1 and 2 were compared with the compounds methyl-7-nitrocoumarin carboxylate and 7-nitrocoumarin-3-carboxylic acid in the absence of cell culture. Minimal essential media was equilibrated to 37° C., 5% CO₂ for 2 hr and compounds were added to a final concentration of 300 μM. The fluorescence signal was monitored at 355/460 over a 7.5 hour time frame (FIG. 11). Both the methyl-7-nitrocoumarin carboxylate and 7-nitrocoumarin-3-carboxylic acid compounds exhibited instability in culture media with a 4.2-fold and 5.3-fold increase in background florescence over 7.8 hours. In contrast, compounds 1 and 2 were significantly more stable over this time period, with a 1.4-fold increase in background florescence over 7.8 hours.

### Example 6

### Superior Anaerobic Stability of Compounds 1-15 Over the 7-nitrocoumarins and 6-chloro-9-nitro-5H-benzo[a]phenoxazin-5-one

Compounds 1-15 or methyl 7-nitrocoumarin carboxylate (methyl 7-nitro-2-oxo-2H-chromene-3-carboxylate) or 7-nitrocoumarin-3-carboylic acid or 6-chloro-9-nitro-5H-benzo[a]phenoxazin-5-one were added to a final concentration of 100 μM in cell-free culture media and were held at 37° C., 5% CO₂ for 4 hr under either air or 95% N₂. The fluorescence signal was monitored at 355/460 except for compounds 4 and 11 that were monitored at 405/585 and compounds 8, 10 and 6-Cl-9-nitro-5-oxo-5H-benzo[a]phenoxazine that were monitored at 355/585, 355/535 and 530/585 respectively (FIG. 12). For compounds 1-15 no detectable fluorescence (above background) was observed under either oxic or anoxic conditions. In contrast, the methyl 7-nitrocoumarin carboxylate compound, the 7-nitrocoumarin-3-carboylic acid compound and the 6-chloro-9-nitro-5H-benzo[a]phenoxazin-5-one compound generated measurable fluorescence in cell-free culture media, specifically under conditions of low oxygen, indicative of instability. Therefore compounds 1-15 are demonstrably superior for detecting mammalian anaerobic reductase activities to methyl 7-nitrocoumarin carboxylate (methyl 7-nitro-2-oxo-2H-chromene-3-carboxylate) and 7-nitrocoumarin-3-carboylic acid as disclosed in US20020031795A1, and 6-chloro-9-nitro-5H-benzo[a]phenoxazin-5-one (also known as C22220, CNOB) as disclosed by Invitrogen (Molecular Probes Handbook, 10^{th} Edition, page 535).

### Example 7

### Suitability of Compounds for Use as Multiplexed Fluorescent Reporter Molecules

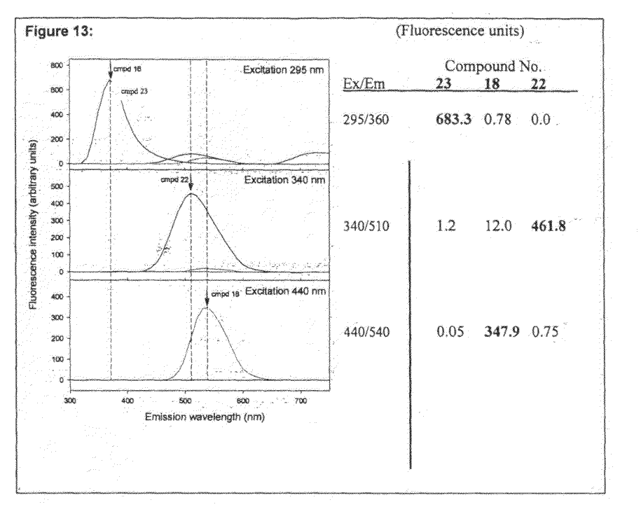

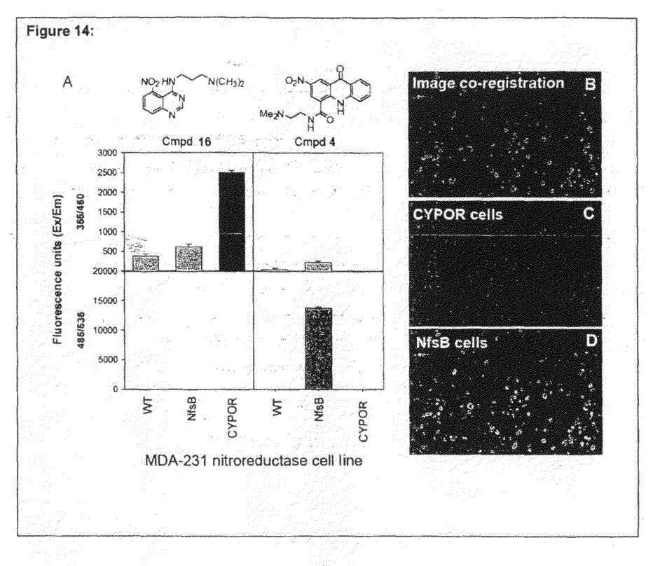

Compounds 23, 18 and 22 (as representative fluorescent amino-substituted aromatic compounds of the present invention) were utilized to exemplify the capacity to specifically and independently monitor multiple fluorescent signal outputs from a mixture of compounds within a sample. Compounds 23, 18 and 22 were placed in 100 mM phosphate buffer (pH 7.0) at 10 μM and subjected to excitation at wavelengths 295, 340 and 440 nm. Fluorescent emission was recorded from 300 to 750 nm for each excitation range (FIG. 13). When the data were collated it was evident that the fluorescent emission maxima of each compound could be independently quantified without interference from the other fluorescent molecules. Specifically, compounds 23, 18 and 22 gave fluorescent output at Ex/Em wavelengths of 295/370=683.3, 0.78, and 0.0 fluorescent units, respectively; at Ex/Em wavelengths of 340/510=1.2, 12.0 and 461.8 fluorescent units, respectively; at Ex/Em wavelengths of 440/540=0.05, 347.9 and 0.75 fluorescent units, respectively. This demonstrated that multiple independent outputs can be recorded from a single sample providing appropriate excitation and emission wavelengths are utilized. The nature of the output may enable co-registration of independent signals within a single test environment which may be correlated to specific mammalian or microbial nitroreductase activities within the test sample of interest.

### Example 8

### Use of Compounds for Multiplexed Reporter Gene Applications

Compounds 4 and 16 (as representative nitro-substituted aromatic compounds of the present invention) were utilized to exemplify the ability to detect individual cell populations. Firstly, to demonstrate selectivity in a 96-well format, fluorescent platereader analyses were performed using MDA-231 wild-type cells (WT) or clones stably expressing either Escherichia coli nfsB (NfsB) or human cytochrome P450 reductase (CYPOR). 30,000 cells were seeded into a plastic 96-well plate in a volume of 0.1 mL. After 2 h attachment samples were incubated with either 200 μM 16 or 100 μM 4 by dilution of DMSO stock solutions into culture medium and addition of 0.1 mL directly into wells. Plates were incubated for 4 h before being read on a fluorescent platereader at Ex/Em 355/460 and 485/535. Values represent the fluorescence in the presence of cells—blank (fluorescence in the absence of cells). When the data were collated it was evident that the fluorescent output of each cell population could be identified with minimal interference from the other reductases-generated fluorescent molecules. Specifically, compounds 4 and 16 gave fluorescent output at Ex/Em wavelengths of 485/535 and 355/460 respectively; detecting the presence of Escherichia coli nfsB and human cytochrome P450 reductase (CYPOR) enzyme activity independently (FIG. 14a). To establish the single cell specificity of compounds 4 and 16 for detection of activities in a mixed (co-cultured) cell population, cells were examined by fluorescent microscopy following concurrent exposure to compounds 4 and 16. MDA-231 cells expressing Escherichia coli nfsB and human cytochrome P450 reductase were mixed 1:1, seeded into a glass 96-well plate (15,000 cells/well in 0.1 mL αMEM+10% FCS+P/S) and allowed to attach overnight. Compound 16 and 4 were diluted into αMEM (from DMSO stock solutions) and 0.1 mL was added to achieve a final concentration of 300 μM and 10 μM respectively. Samples were incubated at 37° C. for 45 minutes, washed three times in PBS and images were acquired on a Nikon TE-2000 inverted fluorescence microscope. FIG. 14b provides a co-registration image of compounds 16 and 4 (DAPI and FITC filter set respectively). The fluorescent emissions of compounds 16 and 14 appear as blue and green marks on the co-registration image. FIGS. 14c and 14d identify each individual cell population, with overlays of 16 and 4 respectively with the corresponding phase contrast image (note: FIG. 14c shows blue fluorescence emission, while FIG. 14d shows green fluorescence emission. This demonstrates that two co-cultured cell populations can be readily identified and distinguished as mutually exclusive cell populations in a common environment through the co-application of two representative nitro-substituted aromatic compounds of the present invention (FIG. 14b-d). This illustrates that multiple independent outputs can be recorded from a single sample providing appropriate excitation and emission wavelengths are utilized. The nature of the output may enable co-registration of independent signals within a single test environment which may be correlated to specific mammalian or microbial nitroreductase activities within the test sample of interest.

### Example 9

### Use of Compounds for Multiplexed Reporter Gene Applications

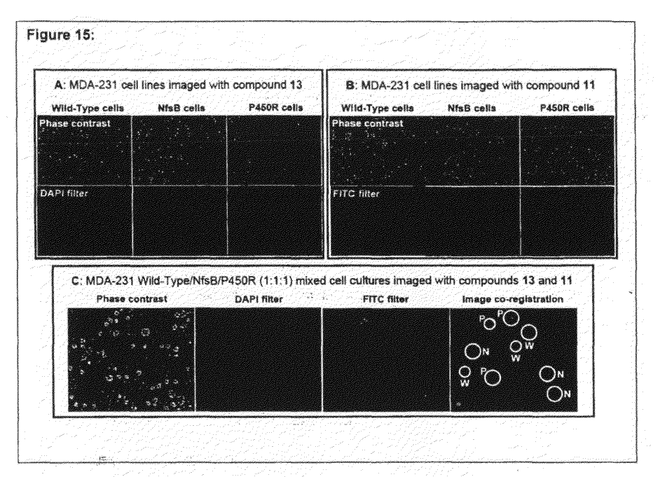

Compounds 11 and 13 (as representative nitro-substituted aromatic compounds of the present invention) were utilized to exemplify the ability to detect individual cell populations. Firstly, to demonstrate selectivity, fluorescent microscope images were captured for each of compound 11 (FIG. 15a) and 13 (FIG. 15b) using appropriate filter settings following exposure of either MDA-231 wild-type cells (WT) or clones stably expressing either Escherichia coli nfsB (NfsB) or human cytochrome P450 reductase (CYPOR). When the data were collated it was evident that the fluorescent output of each cell population could be identified with minimal interference from the other reductases-generated fluorescent molecules. Specifically, compounds 11 and 13 gave fluorescent output at Ex/Em wavelengths of 485/535 and 355/460 respectively; detecting the presence of Escherichia coli nfsB and human cytochrome P450 reductase (CYPOR) enzyme activities. To establish the single cell specificity of compounds 11 and 13 for detection of activities in a mixed (co-cultured) cell population, cells were examined by fluorescent microscopy following concurrent exposure to compounds 11 and 13. MDA-231 wild-type cells or cells expressing Escherichia coli nfsB and human cytochrome P450 reductase were mixed 1:1:1, seeded onto glass coverslips (15,000 cells/well in 0.1 mL αMEM+10% FCS+P/S) and allowed to attach overnight. Compound 11 and 13 were diluted into αMEM (from DMSO stock solutions) and 0.1 mL was added to achieve a final concentration of 10 μM and 100 μM respectively. Samples were incubated at 37° C. for 45 minutes, washed three times in PBS and images were acquired on a Nikon TE-2000 inverted fluorescence microscope. FIG. 15c provides a co-registration image of compounds 11 and 13 (FITC and DAPI filter set respectively). FIG. 15c identifies each individual cell population, with overlay of the corresponding phase contrast image; wild-type cells (W) or clones stably expressing either Escherichia coli nfsB (N) or human cytochrome P450 reductase (R). This demonstrates that three co-cultured cell populations can be readily identified and distinguished as mutually exclusive cell populations in a common environment through the co-application of two representative nitro-substituted aromatic compounds of the present invention (FIG. 15a-c). This illustrates that multiple independent outputs can be recorded from a single sample providing appropriate excitation and emission wavelengths are utilized. The nature of the output may enable co-registration of independent signals within a single test environment which may be correlated to specific mammalian or microbial nitroreductase activities within the test sample of interest.

Examples 7, 8 and 9 clearly demonstrates that a plurality of fluorescent probes can be detected and identified in a common test environment. Therefore a plurality of probes may be used to identify one or more nitroreductase(s) in a common environment. Thus, multiplex reporter output may be achieved.

### Compounds

3-Nitro-6H-benzo[c]chromen-6-one (12) and 6-aminoindazole (23) were purchased from Aldrich Chemical Company.

The following compounds were prepared according to published procedures:

6-Nitro-4(1H)-quinolinone (1) [Ruche man, A. L.; Kerrigan, J. E.; Li, T-K.; Zhou, N.; Liu, A.; Liu, L. F.; LaVoie, E. J. Nitro and amino substitution within the A-ring of 5H-8,9-dimethoxy-5-(2-N,N-dimethylaminoethyl)dibenzo[c,h][1,6]-naphthyridin-6-ones: influence on topoisomerase I-targeting activity and cytotoxicity. Bioorg. & Med. Chem. 2004, 12(13), 3731-42.]

1-Methyl-6-nitro-4(1H)-quinolinone (2) [Denny, W. A.; Atwell, G. J.; Baguley, B. C.; Cain, B. F. Potential Antitumor Agents. 29. Quantitative Structure-Activity Relationships for the Antileukemic Bisquaternary Ammonium Heterocycles. J. Med. Chem. 1979, 22(2), 134-50.]

2-Methyl-6-nitro-4(1H)-quinolinone (3) [Chen, B.; Huang, X.; Wang, J. A versatile synthesis of 2-alkyl and 2-aryl-4-quinolones. Synthesis, 1987, 5, 482-83.]

N-[2-(Dimethylamino)ethyl]-2-nitro-9-oxo-9,10-dihydro-4-acridinecarboxamide (4) [Chen, Q.; Deady, L. W.; Baguley, B. C.; Denny, W. A. Electron-Deficient DNA-Intercalating Agents as Antitumor Drugs: Aza Analogues of the Experimental Clinical Agent N-[2-(Dimethylamino)ethyl]acridine-4-carboxamide. J. Med. Chem. 1994, 37(5), 593-97.]

6-Nitro-4(3H)-quinazolinone (5) [Morley, J. S.; Simpson, J. C. E. The chemistry of simple heterocyclic systems. Part 1. Reactions of 6- and 7-nitro-4-hydroxyquinazoline and their derivatives. J. Chem. Soc. 1948, 360-66.]

6-Nitro-2-phenyl-4(3H)-quinazolinone (6) [Goerdeler, J.; Sappelt, R. Imidazoline-4,5-diones. I. Chem. Ber. 1967, 100(6), 2064-76.]

5-Nitro-2-phenyl-1H-benzimidazole (7) [Tandon, V. K.; Kumar, M. BF₃.Et₂O promoted one-pot expeditious and convenient synthesis of 2-substituted benzimidazoles and 1,3,5-benzoxadiazepines. Tet. Lett. 2004, 45(21), 4185-87 (and references cited therein).]

2-[4-(Dimethylamino)butyl]-5-nitro-1H-benzo[de]isoquinoline-1,3(2H)-dione (11) was prepared according to the procedure described for the 2- and 3-carbon homologues [Zee-Cheng, R. K. Y.; Cheng, C. C.; N-(Aminoalkyl)imide antineoplastic agents. J. Med. Chem. 1985, 28(9), 1216-22.]

Methyl (6-nitro-4-oxo-1(4H)-quinolinyl)acetate (13) [Hutt, M. P.; MacKellar, F. A.; Identification of quinoline nitration products by NOE. J. Het. Chem. 1984, 21(2), 349-52.]

N¹,N¹-Dimethyl-N³-(7-nitro-4-quinolinyl)-1,3-propanediamine (15) [Denny, W. A.; Atwell, G. J.; Roberts, P. B.; Anderson, R. F.; Boyd, M.; Lock, C. J. L.; Wilson, W. R. Hypoxia-Selective Antitumor Agents. 6. 4-(Alkylamino)nitroquinolines: A New Class of Hypoxia-Selective Cytotoxins. J. Med. Chem. 1992, 35(26), 4832-41.]

N¹,N¹-dimethyl-N³-(5-nitro-4-quinazolinyl)-1,3-propanediamine (16) [Denny, W. A.; Atwell, G. J.; Roberts, P. B.; Anderson, R. F.; Boyd, M.; Lock, C. J. L.; Wilson, W. R. Hypoxia-Selective Antitumor Agents. 6. 4-(Alkylamino)nitroquinolines: A New Class of Hypoxia-Selective Cytotoxins. J. Med. Chem. 1992, 35(26), 4832-41.]

### Preparation of methyl 4-{[(5-nitro-1-naphthyl)sulfonyl]amino}butanoate (8), methyl 4-{[(5-nitro-2-naphthyl)sulfonyl]amino}butanoate (9) and methyl 4-{[(8-nitro-2-naphthyl)sulfonyl]amino}butanoate (10)

To a stirred solution of 1-nitronapthalene (2.1 g, 12.1 mmol) in dichloromethane (50 mL) at 0° C. was added chlorosulfonic acid (5 mL) dropwise. The resulting red solution was allowed to warm to room temperature over 18 hours and then poured onto ice water. The aqueous layer was extracted with ethyl acetate (3 times) and the combined organic extracts were washed with brine then dried over anhydrous sodium sulfate and concentrated under reduced pressure to give a crude mixture of sulfonyl chloride isomers that was used directly. The residue was dissolved in tetrahydrofuran to which methyl 4-aminobutyrate hydrochloride (3.73 g, 24.2 mmol) and excess triethylamine (10 mL) were added. After stirring at room temperature for 1 hour the reaction was diluted with water and acidified with 1N hydrochloric acid. The aqueous layer was extracted with ethyl acetate (3 times) and the combined organic extracts were washed with brine, dried over anhydrous sodium sulfate, concentrated under reduced pressure and chromatographed on silica gel, eluting with hexane/ethyl acetate (2:1), to give at highest R_{f}, methyl 4-{[(5-nitro-1-naphthyl)sulfonyl]amino}butanoate (8) (0.61 g, 14%) as a white solid, m.p. 94-96° C. (Et₂O/hexane); ¹H NMR [(CD₃)₂SO] δ 9.02 (d, J=8.7 Hz, 1H), 8.51 (d, J=8.7 Hz, 1H), 8.40 (dd, J=7.6, 0.9 Hz, 1H), 8.29 (dd, J=7.4, 1.0 Hz, 1H), 8.21 (br s, 1H), 7.92 (m, 2H), 3.48 (s, 3H), 2.85 (t, J=6.9 Hz, 2H), 2.20 (t, J=7.3, 2H), 1.55 (m, 2H); MS found: 353 (M+H), 321 (M+H-MeOH); followed at intermediate R_{f} by methyl 4-{[(5-nitro-2-naphthyl)sulfonyl]amino}butanoate (9) (0.24 g, 6%) as an off-white solid, m.p. 102-104° C. (Et₂O/hexane); ¹H NMR [(CD₃)₂SO] δ 8.66 (d, J=1.9 Hz, 1H), 8.62 (d, J=8.3 Hz, 1H), 8.57 (d, J=9.2 Hz, 1H), 8.49 (dd, J=7.7, 1.1 Hz, 1H), 8.08 (dd, J=9.2, 2.0 Hz, 1H), 7.91 (br s, 1H), 7.87 (t, J=7.9 Hz, 1H), 3.51 (s, 3H), 2.84 (t, J=7.0 Hz, 2H), 2.29 (t, J=7.3, 2H), 1.63 (m, 2H); MS found: 353 (M+H), 321 (M+H-MeOH); followed at lowest R_{f} by methyl 4-{[(8-nitro-2-naphthyl)sulfonyl]amino}butanoate (10) (70 mg, 2%) as an off-white solid, m.p. 109-111° C. (Et₂O/hexane); ¹H NMR [(CD₃)₂SO] δ 8.89 (d, J=1.7 Hz, 1H), 8.50 (m, 2H), 8.40 (d, J=8.7 Hz, 1H), 8.01 (dd, J=8.7, 1.8 Hz, 1H), 7.94 (br s, 1H), 7.90 (t, J=7.8 Hz, 1H), 3.50 (s, 3H), 2.84 (t, J=7.0 Hz, 2H), 2.28 (t, J=7.3, 2H), 1.61 (m, 2H); MS found: 353 (M+H), 321 (M+H-MeOH).

### Preparation of N,N-dimethyl-3-[(7-nitro-4-quinolinyl)oxy]-1-propanamine (14)

To a solution of 7-nitro-4-quinolone [Ruchelman, A. L.; Kerrigan, J. E.; Li, T-K.; Zhou, N.; Liu, A.; Liu, L. F.; LaVoie, E. J. Nitro and amino substitution within the A-ring of 5H-8,9-dimethoxy-5-(2-N,N-dimethylaminoethyl)dibenzo[c,h][1,6]-naphthyridin-6-ones: influence on topoisomerase I-targeting activity and cytotoxicity. Bioorg. & Med. Chem. 2004, 12(13), 3731-42.] (0.30 g, 1.58 mmol) in dimethylformamide (10 mL) was added solid potassium carbonate (0.87 g, 6.32 mmol) followed by N-(3-chloropropyl)-N,N-dimethylamine hydrochloride (0.37 g, 2.37 mmol). The resulting suspension was heated at 80° C. under nitrogen with stirring for 3 hours after which time the temperature was reduced to 50° C. for 18 hours. The reaction was diluted with brine and extracted with ethyl acetate (3 times). The combined organic extracts were washed with brine before being dried over anhydrous sodium sulfate and concentrated under reduced pressure. The residue was then suspended in diethyl ether/hexane (1:1, 80 mL) and the undissolved solid (unreacted starting material) was removed by filtration. The filtrate was then concentrated in vacuo, re-dissolved in methanol (10 mL) and converted to the HCl salt by the addition of 1M anhydrous HCl in methanol (1 mL). Diethyl ether was added to precipitate the HCl salt which was collected by filtration. Reverse phase preparative HPLC (CH₃CN/H₂O/TFA) and concentration of the chromatography fractions under reduced pressure then gave N,N-dimethyl-3-[(7-nitro-4-Quinolinyl)oxy]-1-propanamine trifluoroacetate (14) (156 mg, 25%) as a white powder, m.p. 146-149° C. ¹H NMR [(CD₃)₂SO] δ 9.73 (br s, 1H), 8.96 (d, J=5.3 Hz, 1H), 8.75 (d, J=2.2 Hz, 1H), 8.45 (d, J=9.2 Hz, 1H), 8.28 (dd, J=2.2, 9.2 Hz, 1H), 7.25 (d, J=5.3 Hz, 1H), 4.40 (t, J=5.9 Hz, 3H), 3.36 (m, 3H), 2.87 (s, 6H), 2.28 (m, 3H). Found: C, 48.08; H, 4.62; N, 10.16. C₁₆H₁₈F₃N₃O₅.¾H₂O requires C, 47.71; H, 4.88; N, 10.43. Followed by 1-[3-(dimethylamino)propyl]-7-nitro-4(1H)-quinolinone trifluoroacetate (150 mg, 24%) as a yellow powder, m.p. 188-190° C. ¹H NMR [(CD₃)₂SO] δ 9.61 (br s, 1H), 8.54 (d, J=2.0 Hz, 1H), 8.41 (d, J=8.8 Hz, 1H), 8.15-8.11 (m, 2H), 6.25 (d, J=7.8 Hz, 1H), 4.42 (t, J=7.2 Hz, 3H), 3.15 (m, 3H), 2.77 (s, 6H), 2.15 (m, 3H). Found: C, 49.06; H, 4.54; N, 10.75. C₁₆H₁₈F₃N₃O₅ requires C, 49.36; H, 4.66; N, 10.79.

### General Procedure for the Preparation of Amines of FIG. 2.

To a solution of the nitro precursor in methanol/ethyl acetate (1:1) was added a catalytic amount of 5% Pd—C. The resulting suspension was stirred vigorously over an atmosphere of hydrogen (60 psi) for 2 hours and then filtered through celite and concentrated under reduced pressure. Trituration from diethyl ether/hexane then gave:

6-Amino-1-methyl-4(1H)-quinolinone (17), m.p. 212-214° C. ¹H NMR [(CD₃)₂SO] δ 7.73 (d, J=7.5 Hz, 1H), 7.39 (d, J=9.0 Hz, 1H), 7.30 (d, J=2.7 Hz, 1H), 7.06 (dd, J=9.0, 2.8 Hz, 1H), 5.83 (d, J=7.5 Hz, 1H), 5.30 (s, 2H), 3.73 (s, 3H). MS found: 175 (M+H).

2-Amino-N-[2-(dimethylamino)ethyl]-9-oxo-9,10-dihydro-4-acridinecarboxamide (18), m.p. 198-202° C. ¹H NMR [(CD₃)₂SO] δ 11.73 (br s, 1H), 8.75 (t, J=5.0 Hz, 1H), 8.18 (br d, J=8.5 Hz, 1H), 7.67-7.62 (m, 3H), 7.52 (d, J=2.7 Hz, 1H), 7.21-7.17 (m, 1H), 5.30 (s, 2H), 3.45 (q, J=6.6 Hz, 2H), 2.49 (obsc t, J˜7 Hz, 2H), 2.23 (s, 6H). MS found: 325 (M+H).

5-Amino-2-[4-(dimethylamino)butyl]-1H-benzo[de]isoquinoline-1,3(2H)-dione (19), m.p. 99-101° C. ¹H NMR [(CD₃)₂SO] δ 8.08 (dd, J=7.2, 1.0 Hz, 1H), 8.02 (dd, J=8.4, 0.7 Hz, 1H), 7.97 (d, J=2.3 Hz, 1H), 7.61 (dd, J=7.3, 8.2 Hz, 1H), 7.28 (d, J=2.3 Hz, 1H), 5.97 (s, 2H), 4.02 (t, J=7.3 Hz, 2H), 2.22 (t, J=7.1 Hz, 2H), 2.09 (s, 6H), 1.65-1.58 (m, 2H), 1.47-1.40 (m, 2H). MS found: 312 (M+H).

Methyl 4-{[5-amino-1-naphthyl)sulfonyl]amino}butanoate (20), m.p. 90-92° C. ¹H NMR [(CD₃)₂SO] δ 8.37 (d, J=8.5 Hz, 1H), 8.01 (dd, J=7.3, 1.0 Hz, 1H), 7.71 (d, J=8.5 Hz, 1H), 7.77 (br s, 1H), 7.45 (dd, J=7.4, 8.5 Hz, 1H), 7.36 (dd, J=7.7, 8.4 Hz, 1H), 6.79 (dd, J=7.6, 0.7 Hz, 1H), 5.93 (s, 2H), 3.50 (s, 3H), 2.76 (t, J=6.9 Hz, 2H), 2.20 (t, J=7.4 Hz, 2H), 1.58-1.51 (m, 2H). MS found: 323 (M+H).

Methyl 4-{[5-amino-2-naphthyl)sulfonyl]amino}butanoate (21), m.p. 171-175° C. ¹H NMR [(CD₃)₂SO] δ 8.25 (d, J=8.9 Hz, 1H), 8.18 (d, J=1.9 Hz, 1H), 7.64 (obsc br s, 1H), 7.62 (dd, J=8.9, 1.9 Hz, 1H), 7.34 (t, J=7.9 Hz, 1H), 7.26 (d, J=8.1 Hz, 1H), 6.82 (dd, J=7.5, 1.1 Hz, 1H), 5.91 (s, 2H), 3.51 (s, 3H), 2.78 (t, J=7.0 Hz, 2H), 2.29 (t, J=7.4 Hz, 2H), 1.65-1.58 (m, 2H). MS found: 323 (M+H).

Methyl 4-{[(8-amino-2-naphthyl)sulfonyl]amino}butanoate (22), m.p. 162-167° C. ¹H NMR [(CD₃)₂SO] δ 8.53 (br s, 1H), 7.89 (d, J=8.7 Hz, 1H), 7.67 (dd, J=8.7, 1.8 Hz, 1H), 7.56 (br s, 1H), 7.36 (t, J=7.8 Hz, 1H), 7.15 (d, J=8.1 Hz, 1H), 6.79 (dd, J=7.6, 0.9 Hz, 1H), 6.02 (s, 2H), 3.52 (s, 3H), 2.79 (t, J=7.0 Hz, 2H), 2.29 (t, J=7.4 Hz, 2H), 1.64-1.58 (m, 2H). MS found: 323 (M+H).

It is appreciated that the compounds of the invention may occur in different geometric and enantiomeric forms, and that both pure forms and mixtures of these compounds are included.

Wherein the foregoing description reference has been made to integers having known equivalents thereof, those equivalents are herein incorporated as if individually set forth.

It is to be appreciated that variations and modifications may be made to the invention as described without departing from the spirit and scope of the invention.

Throughout this specification, unless the context requires otherwise, the words “comprise”, “comprising” and the like, are to be construed in an inclusive sense as opposed to an exclusive sense, that is to say, in the sense of “including, but not limited to”.

The reference to any prior art in this specification is not, and should not be taken as, an acknowledgment or any form of suggestion that that prior art forms part of the common general knowledge.

1. A method for the fluorescent detection of nitroreductase activity in at least one intact non-microbial cell using a plurality of fluorogenic probes suitable for use as nitroreductase probes, the method including the step of applying a plurality of probes to a sample and monitoring for the presence of at least one nitroreductase.

2. A method according to claim 1, wherein the a plurality of fluorogenic probes are selected from compounds of formulas I-V defined below: 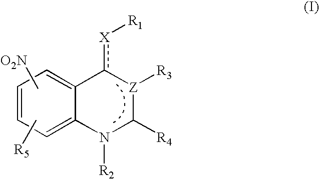
wherein X represents N, NH, NR⁶, O or S;
Z represents C, CH or N;
wherein R¹, if present, may be selected from H, R⁷, (CR⁷R⁸)ₙCOOH, (CR⁷R⁸)ₙCOOR⁹, (CR⁷R⁸)ₙCONH₂, (CR⁷R⁸)ₙCONHR⁹, (CR⁷R⁸)ₙCONR⁹R¹⁰, (CR⁷R⁸)ₙOH, (CR⁷R⁸)ₙOR⁹, (CR⁷R⁸)ₙOPO(OH)₂, COOH, COOR⁷, (CR⁷R⁸)ₙNR⁹R¹⁰, (CR⁷R⁸)ₙ-morpholinyl, (CR⁷R⁸)ₙ-piperazinyl; (CR⁷R⁸)ₙ-1-methylpiperazinyl; (CR⁷R⁸)ₙ-piperidinyl; (CR⁷R⁸)ₙ-pyrrolidinyl or (CR⁷R⁸)ₙ-imidazolyl;
wherein R² may represent H, R¹¹, (C′R¹¹R¹²)ₙCOO(C″R¹¹R¹²)ₙNR¹³R¹⁴, (C′R¹¹R¹²)ₙCONH(C″R¹¹R¹²)ₙNR¹³R¹⁴, wherein C′ and C″ may be optionally and independently substituted with C₁-C₆ alkyl and/or OH;
R³ and R⁴ may independently represent H, R¹⁵, Ar, —(CH═CH)ₙAr; SO₃H, CN
wherein Ar may represent a substituted or unsubstituted phenyl, pyridyl, pyrimidinyl, thiazolyl, oxazoylyl, imidazolyl, furanyl, pyrrolyl, benzoxazolyl, benzthiazolyl, benzofuranyl, indolyl, indazolyl, benzimdazolyl, wherein each Ar is optionally substituted with NO₂, R¹⁶, OH, SH, SR¹⁶, halogen, CF₃, NH₂, NHR¹⁶, NR¹⁶R¹⁷, NHCOR¹⁶, NR¹⁶COR¹⁷, NHCOOR¹⁶, NR¹⁶COOR¹⁷, (CR¹⁶R¹⁷)ₙCOOH, (CR¹⁶R¹⁷)ₙCOOR¹⁸, (CR¹⁶R¹⁷)ₙCONH₂, (CR¹⁶R¹⁷)ₙCONHR¹⁸, (CR¹⁶R¹⁷)ₙCONR¹⁸R¹⁹, (CR¹⁶R¹⁷)ₙOH, (CR¹⁶R¹⁷)ₙOR¹⁸, (CR¹⁶R¹⁷)ₙOPO(OH)₂, COOH, COOR¹⁶, CONH₂, CONHR¹⁶, CONR¹⁶R¹⁷, COR¹⁶, CN, SOR¹⁶, SO₂R¹⁶, SO₂NR¹⁶R¹⁷, SO₃H, or when Z represents C, R³ and R⁴ may together form a fused aromatic ring optionally substituted at one or more of the available carbons with a C₁-C₆ alkyl, halogen, SO₃H or CN;
wherein R⁵ may represent H, C₁-C₆ alkyl, halogen, CN, NO₂, Ar, —(CH═CH)ₙAr, COR²⁰, SOR²⁰, SO₂R²⁰, CO(CR²⁰R²¹)ₙOH, SO(CR²⁰R²¹)ₙOH, SO₂(CR²⁰R²¹)ₙOH, CO(CR²⁰R²¹)ₙCOOR²², SO(CR²⁰R²¹)ₙCOOR²², SO₂(CR²⁰R²¹)COOR²², CO(CR²⁰R²¹)ₙNR²²R²³, SO(CR²⁰R²¹)ₙNR²²R²³, SO₂(CR²⁰R²¹)ₙNR²²R²³, CO(CR²⁰R²¹)ₙOPO(OH)₂, SO(CR²⁰R²¹)ₙOPO(OH)₂, SO₂(CR²⁰R²¹)ₙOPO(OH)₂, CONR²⁰R²¹, SONR²⁰R²¹, SO₂NR²⁰R²¹, CNNR²⁰(CR²¹R²²)ₙCOOR²³, CNNR²⁰(CR²¹R²²)ₙOH, CNNR²⁰(CR²¹R²²)ₙNR²³R²⁴, CNNR²⁰(CR²¹R²²)ₙOPO(OH)₂, CONR²⁰(CR²¹R²²)ₙOH, SONR²⁰(CR²¹R²²)ₙOH, SO₂NR²⁰(CR²¹R²²)ₙOH, CONR²⁰(CR²¹R²²)ₙCOOR²³, SONR²⁰(CR²¹R²²)ₙCOOR²³, SO₂NR²⁰(CR²¹R²²)ₙCOOR²³, CONR²⁰(CR²¹R²²)ₙNR²³R²⁴, SONR²⁰(CR²¹R²²)ₙNR²³R²⁴, SO₂NR²⁰(CR²¹R²²)ₙNR²³R²⁴, CONR²⁰(CR²¹R²²)ₙOPO(OH)₂, SONR²⁰(CR²¹R²²)ₙOPO(OH)₂, SO₂NR²⁰(CR²¹R²²)ₙOPO(OH)₂;
wherein n=0, 1, 2, 3, 4, 5 or 6;
and R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴ independently may represent H, C₁-C₆ alkyl, halogen, OH, (CR²⁵R²⁶)ₙCOOR²⁷, (CR²⁵R²⁶)ₙNR²⁷R²⁸, (CR²⁵R²⁶)ₙOH, (CR²⁵R²⁶)ₙOPO(OH)₂ and may together form a ring selected from morpholinyl, piperazinyl, 1-methylpiperazinyl, piperidinyl, pyrrolidinyl, and imidazolyl, wherein R²⁵, R²⁶, R²⁷ and R²⁸ may represent H, C₁-C₆ alkyl, halogen and may together form a ring selected from morpholinyl, piperazinyl, 1-methylpiperazinyl, piperidinyl, pyrrolidinyl, and imidazolyl; and any pharmaceutically acceptable salt thereof; 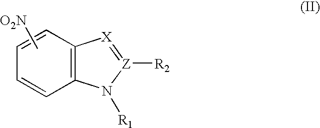
wherein X represents N, CH, O or S;
Z represents C or N;
wherein R¹ and R² if present, may independently represent H, C₁-C₆ alkyl, Ar, (CH═CH)ₙAr, (CR³R⁴)ₙNR⁵R⁶, (CR³R⁴)ₙCOOR⁵, (CR³R⁴)ₙOH, (CR³R⁴)ₙOPO(OH)₂;
wherein n=0, 1, 2, 3, 4, 5 or 6;
wherein Ar may represent a substituted or unsubstituted phenyl, pyridyl, pyrimidinyl, thiazolyl, oxazoylyl, imidazolyl, furanyl, pyrrolyl, benzoxazolyl, benzthiazolyl, benzfuranyl, indolyl, indazolyl, benzimdazolyl, wherein each Ar is optionally substituted with one or more NO₂, CN, R³, OH, OR³, SH, SR³, halogen, CF₃, NH₂, NHR³, NR³R⁴, NHCOR³, NR³COR⁴, NHCOOR³, NR³COOR⁴, (CR³R⁴)ₙCOOH, (CR³R⁴)ₙCOOR⁵, (CR³R⁴)ₙCONH₂, (CR³R⁴)ₙCONHR⁸, (CR³R⁴)ₙCONR⁵R⁶, (CR³R⁴)ₙOH, (CR³R⁴)ₙOR⁵, (CR³R⁴)ₙOPO(OH)₂, COOH, COOR³, CONH₂, CONHR³, CONR³R⁴, COR³, SOR³, SO₂R³, SO₂NR³R⁴, SO₃H;
wherein R³, R⁴, R⁵ or R⁶ may independently represent H, C₁-C₆ alkyl, halogen, OH, (CR⁷R⁸)ₙNR⁹R¹⁰, (CR⁷R⁸)ₙOH, (CR⁷R⁸)ₙOPO(OH)₂ and may together form a ring selected from morpholinyl, piperazinyl, 1-methylpiperazinyl, piperidinyl, pyrrolidinyl, and imidazolyl; wherein R⁷, R⁸, R⁹ and R¹⁰ may represent H or C₁-C₆ alkyl and may together form a ring selected from morpholinyl, piperazinyl, 1-methylpiperazinyl, piperidinyl, pyrrolidinyl, and imidazolyl; and any pharmaceutically acceptable salts thereof; 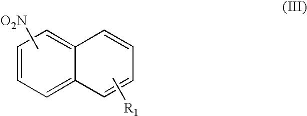
wherein R₁ may represent COR², SOR², SO₂R², CO(CR²R³)ₙOH, SO(CR²R³)ₙOH, SO₂(CR²R³)ₙOH, CO(CR²R³)ₙCOOR⁴, SO(CR²R³)ₙCOOR⁴, SO₂(CR²R³)ₙCOOR⁴, CO(CR²R³)ₙNR⁴R⁵, SO(CR²R³)ₙNR⁴R⁵, SO₂(CR²R³)ₙNR⁴R⁵, CO(CR²R³)ₙOPO(OH)₂, SO(CR²R³)ₙOPO(OH)₂, SO₂(CR²R³)ₙOPO(OH)₂, CONR²R³, SONR²R³, SO₂NR²R³, CNNR²(CR³R⁴)ₙCOOR⁵, CNNR²(CR³R⁴)ₙOH, CNNR²(CR³R⁴)ₙNR⁵R⁶, CNNR²(CR³R⁴)ₙOPO(OH)₂, CONR²(CR³R⁴)ₙOH, SONR²(CR³R⁴)ₙOH, SO₂NR²(CR³R⁴)ₙOH, CONR²(CR³R⁴)ₙCOOR⁵, SONR²(CR³R⁴)ₙCOOR⁵, SO₂NR²(CR³R⁴)ₙCOOR⁵, CONR²(CR³R⁴)ₙNR⁵R⁶, SONR²(CR³R⁴)ₙNR⁵R⁶, SO₂NR²(CR³R⁴)ₙNR⁵R⁶, CONR²(CR³R⁴)ₙOPO(OH)₂, SONR²(CR³R⁴)ₙOPO(OH)₂, SO₂NR²(CR³R⁴)ₙOPO(OH)₂;
wherein n=0, 1, 2, 3, 4, 5 or 6;
and R², R³, R⁴, R⁵, R⁶ independently may represent H, C₁-C₆ alkyl, halogen, OH, (CR⁷R⁸)ₙNR⁹R¹⁰, (CR⁷R⁸)ₙOH, (CR⁷R⁸)ₙOPO(OH)₂ and may together form a ring selected from morpholinyl, piperazinyl, 1-methylpiperazinyl, piperidinyl, pyrrolidinyl, and imidazolyl; wherein R⁷, R⁸, R⁹ and R¹⁰ may represent H, C₁-C₆ alkyl, halogen and may together form a ring selected from morpholinyl, piperazinyl, 1-methylpiperazinyl, piperidinyl, pyrrolidinyl, and imidazolyl; and any pharmaceutically acceptable salt thereof; 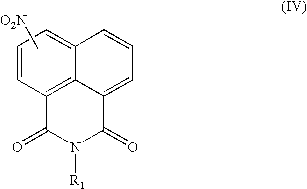
wherein R¹, may be selected from H, R², (CR²R³)ₙCOOH, (CR²R³)ₙCOOR⁴, (CR²R³)ₙCONH₂, (CR²R³)ₙCONHR⁴, (CR²R³)ₙCONR⁴R⁵, (CR²R³)ₙOH, (CR²R³)ₙOR⁴, (CR²R³)ₙOPO(OH)₂, (CR²R³)ₙNR⁴R⁵, (CR²R³)ₙ-morpholinyl, (CR²R³)ₙ-piperazinyl, (CR²R³)ₙ-1-methylpiperazinyl, (CR²R³)ₙ-piperidinyl, (CR²R³)ₙ-pyrrolidinyl, and (CR²R³)ₙ-imidazolyl; wherein n=0, 1, 2, 3, 4, 5 or 6;
and R², R³, R⁴, R⁵ independently may represent H, C₁-C₆ alkyl, halogen, OH, (CR⁶R⁷)ₙNR⁸R⁹, (CR⁶R⁷)ₙOH, (CR⁶R⁷)ₙOPO(OH)₂ and may together form a ring selected from morpholinyl, piperazinyl, 1-methylpiperazinyl, piperidinyl, pyrrolidinyl, and imidazolyl; wherein R⁶, R⁷, R⁸ and R⁹ may represent H, C₁-C₆ alkyl, halogen and may together form a ring selected from morpholinyl, piperazinyl, 1-methylpiperazinyl, piperidinyl, pyrrolidinyl, and imidazolyl; and any pharmaceutically acceptable salt thereof; 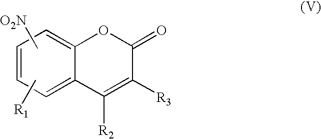
wherein R₁ may represent H, R⁴, COR⁴, SOR⁴, SO₂R⁴, CO(CR⁴R⁵)ₙOH, SO(CR⁴R⁵)ₙOH, SO₂(CR⁴R⁵)ₙOH, CO(CR⁴R⁵)ₙCOOR⁶, SO(CR⁴R⁵)ₙCOOR⁶, SO₂(CR⁴R⁵)ₙCOOR⁶, CO(CR⁴R⁵)ₙNR⁶R⁷, SO(CR⁴R⁵)ₙNR⁶R⁷, SO₂(CR⁴R⁵)ₙNR⁶R⁷, CO(CR⁴R⁵)ₙOPO(OH)₂, SO(CR⁴R⁵)ₙOPO(OH)₂, SO₂(CR⁴R⁵)ₙOPO(OH)₂, CONR⁴R⁵, SONR⁴R⁵, SO₂NR⁴R⁵, CNNR⁴(CR⁵R⁶)ₙCOOR⁷, CNNR⁴(CR⁵R⁶)ₙOH, CNNR⁴(CR⁵R⁶)ₙNR⁷R⁸, CNNR⁴(CR⁵R⁶)ₙOPO(OH)₂, CONR⁴(CR⁵R⁶)ₙOH, SONR⁴(CR⁵R⁶)ₙOH, SO₂NR⁴(CR⁵R⁶)ₙOH, CONR⁴(CR⁵R⁶)ₙCOOR⁷, SONR⁴(CR⁵R⁶)ₙCOOR⁷, SO₂NR⁴(CR⁵R⁶)ₙCOOR⁷, CONR⁴(CR⁵R⁶)ₙNR⁷R⁸, SONR⁴(CR⁵R⁶)ₙNR⁷R⁸, SO₂NR⁴(CR⁵R⁶)ₙNR⁷R⁸, CONR⁴(CR⁵R⁶)ₙOPO(OH)₂, SONR⁴(CR⁵R⁶)ₙOPO(OH)₂, SO₂NR⁴(CR⁵R⁶)ₙOPO(OH)₂;
R² and R³ may independently represent H, R⁹, (CR⁹R¹⁰)ₙCOOH, (CR⁹R¹⁰)ₙCOOR¹¹, (CR⁹R¹⁰)ₙCONH₂, (CR⁹R¹⁰)ₙCONHR¹¹, (CR⁹R¹⁰)ₙCONR¹¹R¹², (CR⁹R¹⁰)ₙOH, (CR⁹R¹⁰)ₙOR¹¹, (CR⁹R¹⁰)ₙOPO(OH)₂, COOH, COOR⁹, CONH₂, CONHR⁹, CONR⁹R¹⁰, COR⁹, CN, SOR⁹, SO₂R⁹, SO₂NR⁹R¹⁰, or R² and R³ may together form a fused aromatic ring optionally substituted at one or more of the available carbons with a C₁-C₆ alkyl, halogen, SO₃H or CN;
wherein n=0, 1, 2, 3, 4, 5 or 6;
and R⁴, R⁵, R⁶, R⁷, R⁸, R⁹, R¹⁰, R¹¹, R¹² independently may represent H, C₁-C₆ alkyl, halogen, OH, (CR¹³R¹⁴)ₙNR¹⁶, (CR¹³R¹⁴)ₙOH, (CR¹³R¹⁴)ₙOPO(OH)₂ and may together form a ring selected from morpholinyl, piperazinyl, 1-methylpiperazinyl, piperidinyl, pyrrolidinyl, and imidazolyl; wherein R¹³, R¹⁴, R¹⁵ and R¹⁶ may represent H, C₁-C₆ alkyl, halogen and may together form a ring selected from morpholinyl, piperazinyl, 1-methylpiperazinyl, piperidinyl, pyrrolidinyl, and imidazolyl; and any pharmaceutically acceptable salt thereof.

3. A method for the fluorescent detection of nitroreductase activity in a least one intact non-microbial cell using at least one fluorogenic probe selected from compounds of formulas I-V as defined below: 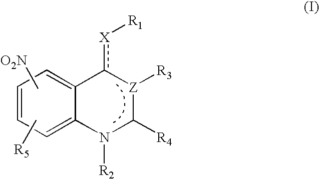
wherein X represents N, NH, NR⁶, O or S;
Z represents C, CH or N;
wherein R¹, if present, may be selected from H, R⁷, (CR⁷R⁸)ₙCOOH, (CR⁷R⁸)ₙCOOR⁹, (CR⁷R⁸)ₙCONH₂, (CR⁷R⁸)ₙCONHR⁹, (CR⁷R⁸)ₙCONR⁹R¹⁰, (CR⁷R⁸)ₙOH, (CR⁷R⁸)ₙOR⁹, (CR⁷R⁸)ₙOPO(OH)₂, COOH, COOR⁷, (CR⁷R⁸)ₙNR⁹R¹⁰, (CR⁷R⁸)ₙ-morpholinyl, (CR⁷R⁸)ₙ-piperazinyl; (CR⁷R⁸)ₙ-1-methylpiperazinyl; (CR⁷R⁸)ₙ-piperidinyl; (CR⁷R⁸)ₙ-pyrrolidinyl or (CR⁷R⁸)ₙ-imidazolyl;
wherein R² may represent H, R¹¹, (C′ R¹¹R¹²)ₙCOO(C″R¹¹R¹²)ₙNR¹³R¹⁴, (C′R¹¹R¹²)ₙCONH(C″R¹¹R¹²)ₙNR¹³R¹⁴, wherein C′ and C″ may be optionally and independently substituted with C₁-C₆ alkyl and/or OH;
R³ and R⁴ may independently represent H, R¹⁵, Ar, —(CH═CH)ₙAr; SO₃H, CN wherein Ar may represent a substituted or unsubstituted phenyl, pyridyl, pyrimidinyl, thiazolyl, oxazoylyl, imidazolyl, furanyl, pyrrolyl, benzoxazolyl, benzthiazolyl, benzofuranyl, indolyl, indazolyl, benzimdazolyl, wherein each Ar is optionally substituted with NO₂, R¹⁶, OH, OR¹⁶, SH, SR¹⁶, halogen, CF₃, NH₂, NHR¹⁶, NR¹⁶R¹⁷, NHCOR¹⁶, NR¹⁶COR¹⁷, NHCOOR¹⁶, NR¹⁶COOR¹⁷, (CR¹⁶R¹⁷)ₙCOOH, (CR¹⁶R¹⁷)ₙCOOR¹⁸, (CR¹⁶R¹⁷)ₙCONH₂, (CR¹⁶R¹⁷)ₙCONHR¹⁸, (CR¹⁶R¹⁷)ₙCONR¹⁸R¹⁹, (CR¹⁶R¹⁷)ₙOH, (CR¹⁶R¹⁷)ₙOR¹⁸, (CR¹⁶R¹⁷)ₙOPO(OH)₂, COOH, COOR¹⁶, CONH₂, CONHR¹⁶, CONR¹⁶R¹⁷, COR¹⁶, CN, SOR¹⁶, SO₂R¹⁶, SO₂NR¹⁶R¹⁷, SO₃H,
wherein R⁵ may represent H, C₁-C₆ alkyl, halogen, CN, NO₂, Ar, —(CH═CH)ₙAr, COR²⁰, SOR²⁰, SO₂R²⁰, CO(CR²⁰R²¹)ₙOH, SO(CR²⁰R²¹)ₙOH, SO₂(CR²⁰R²¹)ₙOH, CO(CR²⁰R²¹)ₙCOOR²², SO(CR²⁰R²¹)ₙCOOR²², SO₂(CR²⁰R²¹)ₙCOOR²², CO(CR²⁰R²¹)ₙNR²²R²³, SO(CR²⁰R²¹)ₙNR²²R²³, SO₂(CR²⁰R²¹)ₙNR²²R²³, CO(CR²⁰R²¹)ₙOPO(OH)₂, SO(CR²⁰R²¹)ₙOPO(OH)₂, SO₂(CR²⁰R²¹)ₙOPO(OH)₂, CONR²⁰R²¹, SONR²⁰R²¹, SO₂NR²⁰R²¹, CNNR²⁰(CR²¹R²²)ₙCOOR²³, CNNR²⁰ (CR²¹R²²)ₙOH, CNNR²⁰(CR²¹R²²)ₙNR²³R²⁴, CNNR²⁰(CR²¹R²²)ₙOPO(OH)₂, CONR²⁰(CR²¹R²²)ₙOH, SONR²⁰(CR²¹R²²)ₙOH, SO₂NR²⁰(CR²¹R²²)ₙOH, CONR²⁰(CR²¹R²²)ₙCOOR²³, SONR²⁰(CR²¹R²²)ₙCOOR²³, SO₂NR²⁰(CR²¹R²²)ₙCOOR²³, CONR²⁰(CR²¹R²²)ₙNR²³R²⁴, SONR²⁰(CR²¹R²²)ₙNR²³R²⁴, SO₂NR²⁰(CR²¹R²²)ₙNR²³R²⁴, CONR²⁰(CR²¹R²²)ₙOPO(OH)₂, SONR²⁰(CR²¹R²²)ₙOPO(OH)₂, SO₂NR²⁰(CR²¹R²²)ₙOPO(OH)₂;
wherein n=0, 1, 2, 3, 4, 5 or 6;
and R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴ independently may represent H, C₁-C₆ alkyl, halogen, OH, (CR²⁵R²⁶)ₙCOOR²⁷, (CR²⁵R²⁶)ₙNR²⁷R²⁸, (CR²⁵R²⁶)ₙOH, (CR²⁵R²⁶)ₙOPO(OH)₂ and may together form a ring selected from morpholinyl, piperazinyl, 1-methylpiperazinyl, piperidinyl, pyrrolidinyl, imidazolyl, wherein R²⁵, R²⁶, R²⁷ and R²⁸ may represent H, C₁-C₆ alkyl, halogen and may together form a ring selected from morpholinyl, piperazinyl, 1-methylpiperazinyl, piperidinyl, pyrrolidinyl, and imidazolyl; and any pharmaceutically acceptable salt thereof; 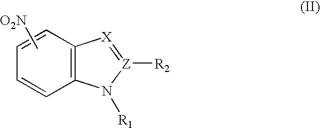
wherein X represents N, CH, O or S;
Z represents C or N;
wherein R¹ and R² if present, may independently represent H, C₁-C₆ alkyl, Ar, (CH═CH)ₙAr, (CR³R⁴)ₙNR⁵R⁶, (CR³R⁴)ₙCOOR⁵, (CR³R⁴)ₙOH, (CR³R⁴)ₙOPO(OH)₂;
wherein n=0, 1, 2, 3, 4, 5 or 6;
wherein Ar may represent a substituted or unsubstituted phenyl, pyridyl, pyrimidinyl, thiazolyl, oxazoylyl, imidazolyl, furanyl, pyrrolyl, benzoxazolyl, benzthiazolyl, benzfuranyl, indolyl, indazolyl, benzimdazolyl, wherein each Ar is optionally substituted with one or more NO₂, CN, R³, OH, OR³, SH, SR³, halogen, CF₃, NH₂, NHR³, NR³R⁴, NHCOR³, NR³COR⁴, NHCOOR³, NR³COOR⁴, (CR³R⁴)ₙCOOH, (CR³R⁴)ₙCOOR⁵, (CR³R⁴)ₙCONH₂, (CR³R⁴)ₙCONHR⁵, (CR³R⁴)ₙCONR⁵R⁶, (CR³R⁴)ₙOH, (CR³R⁴)ₙOR⁵, (CR³R⁴)ₙOPO(OH)₂, COOH, COOR³, CONH₂, CONHR³, CONR³R⁴, COR³, SOR³, SO₂R³, SO₂NR³R⁴, SO₃H;
wherein R³, R⁴, R⁵ or R⁶ may independently represent H, C₁-C₆ alkyl, halogen, OH, (CR⁷R⁸)ₙNR⁹R¹⁰, (CR⁷R⁸)ₙOH, (CR⁷R⁸)ₙOPO(OH)₂ and may together form a ring selected from morpholinyl, piperazinyl, 1-methylpiperazinyl, piperidinyl, pyrrolidinyl, and imidazolyl; wherein R⁷, R⁸, R⁹ and R¹⁰ may represent H or C₁-C₆ alkyl and may together form a ring selected from morpholinyl, piperazinyl, 1-methylpiperazinyl, piperidinyl, pyrrolidinyl, and imidazolyl; and any pharmaceutically acceptable salts thereof; 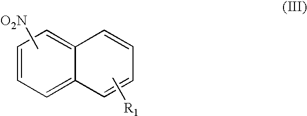
wherein R₁ may represent COR², SOR², SO₂R², CO(CR²R³)ₙOH, SO(CR²R³)ₙOH, SO₂(CR²R³)ₙOH, CO(CR²R³)ₙCOOR⁴, SO(CR²R³)ₙCOOR⁴, SO₂(CR²R³)ₙCOOR⁴, CO(CR²R³)ₙNR⁴R⁵, SO(CR²R³)ₙNR⁴R⁵, SO₂(CR²R³)ₙNR⁴R⁵, CO(CR²R³)ₙOPO(OH)₂, SO(CR²R³)ₙOPO(OH)₂, SO₂(CR²R³)ₙOPO(OH)₂, CONR²R³, SONR²R³, SO₂NR²R³, CNNR²(CR³R⁴)ₙCOOR⁵, CNNR²(CR³R⁴)ₙOH, CNNR²(CR³R⁴)ₙNR⁵R⁶, CNNR²(CR³R⁴)ₙOPO(OH)₂, CONR²(CR³R⁴)ₙOH, SONR²(CR³R⁴)ₙOH, SO₂NR²(CR³R⁴)ₙOH, CONR²(CR³R⁴)ₙCOOR⁵, SONR²(CR³R⁴)ₙCOOR⁵, SO₂NR²(CR³R⁴)ₙCOOR⁵, CONR²(CR³R⁴)ₙNR⁵R⁶, SONR²(CR³R⁴)ₙNR⁵R⁶, SO₂NR²(CR³R⁴)ₙNR⁵R⁶, CONR²(CR³R⁴)ₙOPO(OH)₂, SONR²(CR³R⁴)ₙOPO(OH)₂, SO₂NR²(CR³R⁴)ₙOPO(OH)₂;
wherein n=0, 1, 2, 3, 4, 5 or 6;
and R², R³, R⁴, R⁵, R⁶ independently may represent H, C₁-C₆ alkyl, halogen, OH, (CR⁷R⁸)ₙNR⁹R¹⁰, (CR⁷R⁸)ₙOH, (CR⁷R⁸)ₙOPO(OH)₂ and may together form a ring selected from morpholinyl, piperazinyl, 1-methylpiperazinyl, piperidinyl, pyrrolidinyl, and imidazolyl; wherein R⁷, R⁸, R⁹ and R¹⁰ may represent H, C₁-C₆ alkyl, halogen and may together form a ring selected from morpholinyl, piperazinyl, 1-methylpiperazinyl, piperidinyl, pyrrolidinyl, and imidazolyl; and any pharmaceutically acceptable salt thereof; 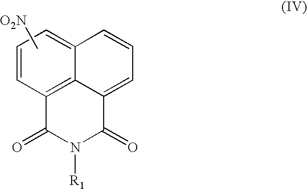
wherein R¹, may be selected from H, R², (CR²R³)ₙCOOH, (CR²R³)ₙCOOR⁴, (CR²R³)ₙCONH₂, (CR²R³)ₙCONHR⁴, (CR²R³)ₙCONR⁴R⁵, (CR²R³)ₙOH, (CR²R³)ₙOR⁴, (CR²R³)ₙOPO(OH)₂, (CR²R³)ₙNR⁴R⁵, (CR²R³)ₙ-morpholinyl, (CR²R³)ₙ-piperazinyl; (CR²R³)ₙ-1-methylpiperazinyl, (CR²R³)ₙ-piperidinyl, (CR²R³)ₙ-pyrrolidinyl, and (CR²R³)ₙ-imidazolyl; wherein n=0, 1, 2, 3, 4, 5 or 6;
and R², R³, R⁴, R⁵ independently may represent H, C₁-C₆ alkyl, halogen, OH, (CR⁶R⁷)ₙNR⁸R⁹, (CR⁶R⁷)ₙOH, (CR⁶R⁷)ₙOPO(OH)₂ and may together form a ring selected from morpholinyl, piperazinyl, 1-methylpiperazinyl, piperidinyl, pyrrolidinyl, and imidazolyl; wherein R⁶, R⁷, R⁸ and R⁹ may represent H, C₁-C₆ alkyl, halogen and may together form a ring selected from morpholinyl, piperazinyl, 1-methylpiperazinyl, piperidinyl, pyrrolidinyl, and imidazolyl; and any pharmaceutically acceptable salt thereof; 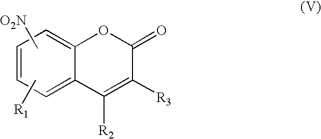
wherein R₁ may represent H, R⁴, COR⁴, SOR⁴, SO₂R⁴, CO(CR⁴R⁵)ₙOH, SO(CR⁴R⁵)ₙOH, SO₂(CR⁴R⁵)ₙOH, CO(CR⁴R⁵)ₙCOOR⁶, SO(CR⁴R⁵)ₙCOOR⁶, SO₂(CR⁴R⁵)ₙCOOR⁶, CO(CR⁴R⁵)ₙNR⁶R⁷, SO(CR⁴R⁵)ₙNR⁶R⁷, SO₂(CR⁴R⁵)ₙNR⁶R⁷, CO(CR⁴R⁵)ₙOPO(OH)₂, SO(CR⁴R⁵)ₙOPO(OH)₂, SO₂(CR⁴R⁵)ₙOPO(OH)₂, CONR⁴R⁵, SONR⁴R⁵, SO₂NR⁴R⁵, CNNR⁴(CR⁵R⁶)ₙCOOR⁷, CNNR⁴(CR⁵R⁶)ₙOH, CNNR⁴(CR⁵R⁶)ₙNR⁷R⁸, CNNR⁴(CR⁵R⁶)ₙOPO(OH)₂, CONR⁴(CR⁵R⁶)ₙOH, SONR⁴(CR⁵R⁶)ₙOH, SO₂NR⁴(CR⁵R⁶)ₙOH, CONR⁴(CR⁵R⁶)ₙCOOR⁷, SONR⁴(CR⁵R⁶)ₙCOOR⁷, SO₂NR⁴(CR⁵R⁶)ₙCOOR⁷, CONR⁴(CR⁵R⁶)ₙNR⁷R⁸, SONR⁴(CR⁵R⁶)ₙNR⁷R⁸, SO₂NR⁴(CR⁵R⁶)ₙNR⁷R⁸, CONR⁴(CR⁵R⁶)ₙOPO(OH)₂, SONR⁴(CR⁵R⁶)ₙOPO(OH)₂, SO₂NR⁴(CR⁵R⁶)ₙOPO(OH)₂;
R² and R³ may independently represent H, R⁹, (CR⁹R¹⁰)ₙCOOH, (CR⁹R¹⁰)ₙCOOR¹¹, (CR⁹R¹⁰)ₙCONH₂, (CR⁹R¹⁰)ₙCONHR¹¹, (CR⁹R¹⁰)ₙCONR¹¹R¹², (CR⁹R¹⁰)ₙOH, (CR⁹R¹⁰)ₙOR¹¹, (CR⁹R¹⁰)ₙOPO(OH)₂, COOH, COOR⁹, CONH₂, CONHR⁹, CONR⁹R¹⁰, COR⁹, CN, SOR⁹, SO₂R⁹, SO₂NR⁹R¹⁰, or R² and R³ may together form a fused aromatic ring optionally substituted at one or more of the available carbons with a C₁-C₆ alkyl, halogen, SO₃H or CN;
wherein n=0, 1, 2, 3, 4, 5 or 6;
and R⁴, R⁵, R⁶, R⁷, R⁸, R⁹, R¹⁰, R¹¹, R¹² independently may represent H, C₁-C₆ alkyl, halogen, OH, (CR¹³R¹⁴)ₙNR¹⁵R¹⁶, (CR¹³R¹⁴)ₙOH, (CR¹³R¹⁴)ₙOPO(OH)₂ and may together form a ring selected from morpholinyl, piperazinyl, 1-methylpiperazinyl, piperidinyl, and pyrrolidinyl, imidazolyl; wherein R¹³, R¹⁴, R¹⁵ and R¹⁶ may represent H, C₁-C₆ alkyl, halogen and may together form a ring selected from morpholinyl, piperazinyl, 1-methylpiperazinyl, piperidinyl, pyrrolidinyl, imidazolyl; and any pharmaceutically acceptable salt thereof;
the method including the step of applying at least one fluorescent probe to a sample and monitoring for the presence of at least one nitroreductase.

4. A method as defined in claim 2, wherein the fluorogenic probes are selected from compounds of formula I.

5. A method according to claim 4, wherein X is O or NH, Z is CH or NH and R³ is H.

6. A method according to claim 5, wherein the compound of formula I is selected from:
6-nitro-4(1H)-quinolinone;
1-methyl-6-nitro-4(1H)-quinolinone;
2-methyl-6-nitro-4(1H)-quinolinone;
N-[2-(dimethylamino)ethyl]-2-nitro-9-oxo-9,10-dihydro-4-acridinecarboxamide;
6-nitro-4(3H)-quinazolinone;
6-nitro-2-phenyl-4(3H)-quinazolinone;
methyl (6-nitro-4-oxo-1(4H)-quinolinyl)acetate;
N,N-dimethyl-3-[(7-nitro-4-quinolinyl)oxy]-1-propanamine;
N¹,N¹-dimethyl-N³-(7-nitro-4-quinolinyl)-1,3-propanediamine;
N¹,N¹-dimethyl-N³-(5-nitro-4-quinazolinyl)-1,3-propanediamine;
or any pharmaceutically acceptable salt thereof.

7. A method as defined in claim 3 wherein the fluorogenic probe(s) is selected from compounds of formula I.

8. A method according to claim 7 wherein X is O or NH, Z is CH or NH and R³ is H.

9. A method according to claim 7 wherein the compound of formula I is selected from:
6-nitro-4(1H)-quinolinone;
1-methyl-6-nitro-4(1H)-quinolinone;
2-methyl-6-nitro-4(1H)-quinolinone;
6-nitro-4(3H)-quinazolinone;
6-nitro-2-phenyl-4(3H)-quinazolinone;
methyl (6-nitro-4-oxo-1(4H)-quinolinyl)acetate;
N,N-dimethyl-3-[(7-nitro-4-quinolinyl)oxy]-1-propanamine;
N¹,N¹-dimethyl-N³-(7-nitro-4-quinolinyl)-1,3-propanediamine;
N¹,N¹-dimethyl-N³-(5-nitro-4-quinazolinyl)-1,3-propanediamine;
any pharmaceutically acceptable salt thereof.

10. A method as defined in claim 2, wherein the fluorogenic probes are selected from compounds of formula II.

11. A method according to claim 10, wherein X is N and Z is C, R¹=H and R²=Ar.

12. A method according to claim 10, wherein the compound of formula II is 5-nitro-2-phenyl-1H-benzimidazole or any pharmaceutically acceptable salt thereof.

13. A method as defined in claim 2, wherein the fluorogenic probes are selected from compounds of formula III.

14. A method according to claim 13, wherein R¹ is SO₂NR²(CR³CR⁴)ₙCOOR⁵, wherein R², R³, R⁴ and R⁵, may represent H, C₁-C₆ alkyl, halogen or OH and wherein n=0, 1, 2, 3, 4, 5 or 6.

15. A method according to claim 13, wherein the compound of formula III is selected from:
methyl 4-{[(5-nitro-1-naphthyl)sulfonyl]amino}butanoate;
methyl 4-{[(5-nitro-2-naphthyl)sulfonyl]amino}butanoate;
methyl 4-{[(8-nitro-2-naphthyl)sulfonyl]amino}butanoate;
any pharmaceutically acceptable salt thereof.

16-21. (canceled)

22. A method as defined in claim 1, wherein the at least one nitroreductase is a human oxidoreductase.

23. A method as defined in claim 22, wherein the human oxidoreductase is selected from known human enzymes classified as EC1.

24. A method as defined in claim 22, wherein the nitroreductase is selected from:
EC 1.1: which includes oxidoreductases that act on the CH—OH group of donors,
EC 1.2: which includes oxidoreductases that act on the aldehyde or oxo group of donors,
EC 1.3: which includes oxidoreductases that act on the CH—CH group of donors,
EC 1.4: which includes oxidoreductases that act on the CH—NH₂ group of donors,
EC 1.5: which includes oxidoreductases that act on CH—NH group of donors,
EC 1.6: which includes oxidoreductases that act on NADH or NADPH,
EC 1.7: which includes oxidoreductases that act on other nitrogenous compounds as donors,
EC 1.8: which includes oxidoreductases that act on a sulfur group of donors,
EC 1.9: which includes oxidoreductases that act on a heme group of donors,
EC 1.10: which includes oxidoreductases that act on diphenols and related substances as donors,
EC 1.11: which includes oxidoreductases that act on peroxide as an acceptor (peroxidases),
EC 1.12: which includes oxidoreductases that act on hydrogen as donors,
EC 1.13: which includes oxidoreductases that act on single donors with incorporation of molecular oxygen (oxygenases),
EC 1.14: which includes oxidoreductases that act on paired donors with incorporation of molecular oxygen,
EC 1.15: which includes oxidoreductases that act on superoxide radicals as acceptors,
EC 1.16: which includes oxidoreductases that oxidize metal ions,
EC 1.17: which includes oxidoreductases that act on CH or CH₂ groups,
EC 1.18: which includes oxidoreductases that act on iron-sulfur proteins as donors,
EC 1.19: which includes oxidoreductases that act on reduced flavodoxin as a donor,
EC 1.21: which includes oxidoreductases that act on X—H and Y—H to form an X—Y bond, and
EC 1.97: which includes other oxidoreductases.

25. A method as defined in claim 1, wherein the at least one nitroreductase is a bacterial or fungal nitroreductase.

26. A method as defined in claim 25, wherein the at least one bacterial nitroreductase is selected from type I nitroflavin reductase NfsA and NfsB superfamilies, NQO1-like and YieF-like nitroreductase enzymes, and putative nitroreductase genes showing evidence of significant sequence homology thereof.

27. A method for identifying the presence of cellular hypoxia, the method including:
(i) contacting an effective amount of at least one compound of formulas I-V as defined in claim 2, to a sample including at least one non-microbial cell; and
(ii) monitoring for the formation of at least one fluorescent derivative arising from reduction by at least one nitroreductase.

28. (canceled)

29. The method according to claim 27, wherein the nitroreductase is a human nitroreductase.

30. The method according to claim 27, wherein the nitroreductase is selected from:
DT-diaphorase [NQO1; E.C.1.6.99.2];
Cytochrome P450-reductase [CYPOR; E.C.1.6.2.4];
Inducible nitric oxide synthase [NOS2A; E.C.1.14.13.39];
Cytochrome B5 reductase [DIAL; E.C.1.6.2.2];
Xanthine oxidase [XO; E.C.1.17.3.2];
Xanthine dehydrogenase [XDH; E.C.1.17.1.4];
Adrenodoxin oxidoreductase [FDXR; E.C.1.18.1.2];
Methionine synthase reductase [MTRR; E.C.1.16.1.8];
Aldose reductase [ALDR1; E.C.1.1.1.21];
Aldehyde reductase [AKR1B10; E.C.1.1.1.2] and
Thioredoxin reductase [TXNRD; E.C.1.8.1.9].

31. A method as defined in claim 2, wherein the step of monitoring for the presence of nitroreductase includes the step of monitoring for the presence of a reduced fluorescent derivative of at least one compound of formulas I-V.

32. A method as defined in claim 31, wherein the presence of the reduced fluorescent derivative is determined from the fluorescence emission.

33. A method according to claim 32, wherein the reduced derivative(s) may be excited at predetermined wavelengths in the UV/visible range.

34. A method according to claim 33, wherein the excitation wavelength is between 200-700 nm.

35. A method according to claim 33, wherein the excitation wavelength is selected from 295, 340, 355, 405, 440 and 485 nm.

36. A method according to claim 32, wherein the fluorescence emission(s) is in the UV/visible/IR range.

37. A method according to claim 36, wherein the fluorescence emission wavelength is between 300-800 nm

38. A method according to claim 36, wherein the fluorescence emission wavelength is selected from 370, 460, 510, 535, 540 and 585 nm.

39. A method as defined in any one of claim 1, wherein the method further includes the step of quantifying the one or more nitroreductases.

40. A method as defined in claim 39, wherein the quantification of nitroreductase includes the step of quantifying the formation of the fluorescent derivative from the fluorescence emission intensity.

41. A method according to claim 1, wherein the monitoring can be performed in a common test environment.

42. An assay for the detection of one or more nitroreductase including the steps of:
(i) contacting an effective amount of a plurality fluorogenic probes with a sample including at least one non-microbial cell;
(ii) monitoring for the formation of fluorescent derivatives.

43-44. (canceled)

45. An assay according to claim 42, wherein the assay further includes the analytical step of quantifying the formation of at least one fluorescent derivative(s) from the fluorescence emission intensity.

46. An assays for the detection of one or more nitroreductase comprising at least one test environment containing a plurality of fluorogenic probes, wherein a sample including at least one non-microbial cell may be added and the test environment monitored for the formation of fluorescent derivative(s).

47. An assay for the detection of one or more nitroreductase comprising at least one test environment containing a plurality of fluorogenic probes, wherein a sample including at least one non-microbial cell ma be added and the test environment monitored for the formation of fluorescent derivative(s), wherein the fluorogenic probes may be selected from compounds of formulas I-V as defined in claim 2.

48. A method for fluorescent detection of nitroreductase activity in at least one intact non-microbial cell using at least one fluorogenic probe(s) capable of being entrapped in the non-microbial cell selected from compounds of formula I as defined in claim 3,
the method including the step of applying at least one compound of formula I to a sample including at least one non-microbial cell and monitoring for the presence of at least one nitroreductase.

49. A method as defined in claim 48 wherein at least one of the groups R₁, R₂, R₃, R₄ and R₅ of the compounds of formula I comprises a cell membrane permeabilising group.

50. A method as defined in claim 49 wherein the cell membrane permeabilising group is cleaved after entry into the cell.

51. A method as defined in claims 50 wherein the cell membrane permeabilising group is cleaved by hydrolysis after entry into the cell.

52. A method as defined in claim 49 wherein the cell membrane permeabilising group is an ester, amine or ether group.

53. A method as defined in claim 48 wherein the compound of formula I is selected from:
Methyl (6-nitro-4-oxo-1(4H)-quinolinyl)acetate,
N,N-dimethyl-3-[(7-nitro-4-quinolinyl)oxy]-1-propanamine, and
N¹,N¹-dimethyl-N³-(7-nitro-4-quinolinyl)-1,3-propanediamine.

54. A method as defined in claim 48 wherein at least one of the groups R₁, R₂, R₃, R₄ and R₅ of the compounds of formula I comprises a group with DNA affinity.

55. A method as defined in claim 54 wherein the fluorogenic probe is non-microbial cell nuclear localised.

56. A method as defined in claim 48 wherein the fluorescence of the fluorogenic probe is durable and specific to nitroreductase positive cells.

57. A compound of formula III as defined in claim 2, wherein formula III represents one of the following:
methyl 4-{[(5-nitro-1-naphthyl)sulfonyl]amino}butanoate;
methyl 4-{[(5-nitro-2-naphthyl)sulfonyl]amino}butanoate;
methyl 4-{[(8-nitro-2-naphthyl)sulfonyl]amino}butanoate;
or any pharmaceutically acceptable salt thereof.
